(19) United States
(12) Patent Application Publication
(10) Pub. No.: US 2014/0090094 A1
(43) Pub. Date: Mar. 27, 2014

(54) MODULATORS OF SLC22A7

(71) Applicants: Stefan Golz, Muelheim an der Ruhr (DE); Andreas Geerts, Wuppertal (DE); Dirk Gründemann, Bruhl (DE)

(72) Inventors: Stefan Golz, Muelheim an der Ruhr (DE); Andreas Geerts, Wuppertal (DE); Dirk Gründemann, Bruhl (DE)

(73) Assignee: BAYER INTELLECTUAL PROPERTY GmbH, Monheim (DE)

(21) Appl. No.: 14/004,027

(22) PCT Filed: Mar. 5, 2012
(86) PCT No.: PCT/EP2012/053754

§ 371 Date: Dec. 1, 2013

(30) Foreign Application Priority Data
Mar. 9, 2011 (EP) 11157494.3

(51) Int. Cl. G01N 33/68; C12N 15/113; A61K 49/00; C07K 16/28

(52) U.S. Cl. CPC G01N 33/6872; C07K 16/28; C12N 15/1138; A61K 49/0008
USPC 800/3; 530/387.9; 536/24.5; 435/29; 435/7.21; 436/501; 800/9

(57) ABSTRACT

The present invention is directed to the identification of modulators for SLC22A7 transporter and therapeutic uses thereof. Hence, in one embodiment the present invention relates to a method for identifying and/or obtaining a compound capable of modulating glutamate transport, comprising contacting a test compound with a system for measuring those transport activity, which system comprises an SLC22A7 polypeptide or a functional fragment thereof, and a substrate for measuring glutamate transport by the system; and detecting an altered level of the those transport activity of the SLC22A7 polypeptide or functional fragment in the presence of the test compound compared to the described transport activity in the absence of the test compound and/or presence of a control.

### TECHNICAL FIELD OF THE INVENTION

The present invention is in the field of molecular biology, more particularly, the present invention relates to modulators and substrates of SLC22A7 and the use of those.

### BACKGROUND OF THE INVENTION

### SLC Transporter

The solute carrier (SLC) group of membrane transport proteins include over 300 members organized into 47 families. The SLC gene nomenclature system was originally proposed by the Human Genome Organization (HUGO) and is the basis for the official HUGO names of the genes that encode these transporters. A more general transmembrane transporter classification can be found in TCDB database.

Solutes that are transported by the various SLC group members are extraordinarily diverse and include both charged and uncharged organic molecules as well as inorganic ions.

As is typical of integral membrane proteins, SLCs contain a number of hydrophobic transmembrane alpha helices connected to each other by hydrophilic intra- and extra-cellular loops. Depending on the SLC, these transporters are functional as either monomers or obligate homo- or hetero-oligomers.

By convention of the nomenclature system, members within an individual SLC family have greater than 20-25% sequence homology to each other. In contrast, the homology between SLC families is very low to non-existent. Hence the criteria for inclusion of a family into the SLC group is not evolutionary relatedness to other SLC families but rather functional (i.e., an integral membrane protein which transports a solute).

The SLC group includes examples of transport proteins that are:
facilitative transporters (allow solutes to flow downhill with their electrochemical gradients)
secondary active transporters (allow solutes to flow uphill against their electrochemical gradient by coupling to transport of a second solute that flows downhill with its gradient such that the overall free energy change is still favorable)

The SLC series does not include members of transport protein families which have previously been classified by other widely accepted nomenclature systems including:
primary active transporters (allow flow uphill against electrochemical gradients) such as ABC (ATP Binding Cassette) transporters by coupling transport to an energy releasing event such as ATP hydrolysis
ion channels
aquaporins (water channels)

### SLC22A7

Solute carrier family 22 member 7 is a protein that in humans is encoded by the SLC22A7 gene. The protein encoded by this gene is involved in the sodium-independent transport and excretion of organic anions, some of which are potentially toxic. The encoded protein is an integral membrane protein and e.g. appears to be localized to the basolateral membrane of the kidney. Alternatively spliced transcript variants encoding different isoforms have been described.

The integral membrane protein presently called OAT2 (human gene symbol SLC22A7) was first spotted with a monoclonal antibody at the sinusoidal membrane domain of rat hepatocytes. Northern analysis of several rat tissues with the corresponding cDNA as probe revealed strong expression merely in liver and much less in kidney. Since hepatic transcription starts only at birth, OAT2 represents a marker for highly-differentiated liver cells. OAT2 is a member of the SLC22 family of transport proteins. Injection of cRNA of OAT2 from rat into Xenopus oocytes was reported to promote uptake of several radiolabeled substrates of OAT1 (SLC22A6) like salicylate, PAH, and 2-oxoglutarate alias alpha-ketoglutarate; the protein was thus named “multispecific” organic anion transporter type 2. The main and physiologically relevant substrate of SLC22A7 was unknown before.

However, subsequent functional studies with OAT2 from human, rat, and mouse have yielded inconsistent data on the substrate specificity of this carrier. Most of these studies were based on expression in Xenopus oocytes. Since oocytes sometimes generate dramatic artefacts, we consider heterologous expression in mammalian cell lines more reliable. But even with cell lines, binding of radiotracer to the expressed protein may be misinterpreted as uptake. Indeed, with other SLC22 transporters we have been unable to confirm suggested transport activities in several instances. Since OAT2 is not closely related in amino acid sequence to OAT1 (40% identity among human sequences), the function could be quite different. On the whole, to us and others the physiological function of the carrier OAT2 was unsettled.

### Drug Discovery

During the process of drug design, medicinal chemists need to solve three basic problems: lead compound identification; lead optimization elevating the lead into candidate drug status; and, following detailed pharmacological studies, the improvement of pharmacokinetic and pharmacodynamic properties of the future drug. Traditionally, natural products, synthetic compounds, human metabolites, metabolites of drugs, known drugs, analogs of the transition state of enzymatic reactions and suicidal inhibitors of enzymes are used as sources of lead structures. In the past few decades, powerful experimental methods have sped up the search for lead structures. HTS (simultaneous testing in vitro of hundreds and thousands of compounds from libraries of chemical structures) is used for identification of ‘hits’, molecules that strongly bind the selected enzymes or receptors. To become leads these compounds need to have lead-like properties and, subsequently, to confirm their activity in more elaborate biological assays. Another experimental approach makes use of combinatorial chemistry, where tens and hundreds of compounds from building blocks are synthesized in parallel and then tested for activity, using automated systems. Recently, the dynamic combinatorial chemistry has developed quickly, which implies addition of the target enzyme or receptor to the reactive system, thus creating a driving force that favors the formation of the best binding combination of building blocks. This selfscreening process accelerates the identification of lead compounds for drug discovery. If the 3D structure of the biological target is available from X-ray crystallography and the active site is known, methods of structure-based drug design (SBDD) can be applied for lead identification. There are two basic strategies for searching for biologically active compounds by SBDD: molecular database screening and de novo ligand design. During screening, the different compounds from databases are docked to the active site of a target. The docking program generates hypotheses of probable spatial space, is widely used. Analysis of 3D-QSAR models is carried out by using contour maps of different fields, showing favorable and unfavorable regions for ligand interaction. The QSAR modeling methods allow estimating probable pharmacological activity of unknown compounds. The ‘classical’ QSAR is effective for the development of analogues close to the compounds under modeling. The 3D-QSAR methods are capable of predicting the pharmacological activity of compounds from different chemical classes. Converting a drug candidate with good in vitro properties into a drug with sufficient in vivo properties (for example, decrease in toxicity, increase in solubility, chemical stability and biological half-life) is the third stage of the drug design process. The approaches used in this stage include: the introduction of bioisosters; the design of prodrugs transforming themselves into an active form in the body; twin drugs carrying two pharmacophore groups that bind to one molecule; and soft drugs, which have a pharmacological action localized in specific organs (their distribution in other organs gives rise to metabolic destruction or inactivation). However, the identification and development of a substance for therapeutic intervention is often hampered in case of orphan targets, where the physiological substrate is not known.

In view of the above, the technical problem underlying the present invention is the provision of a SLC22A7 transport assay system reflecting the physiological activity of SLC22A7 which allows therapeutic intervention for disorders that are related to the malfunction or the lack of this transporter. The solution is the provision of the natural substrate(s) (e.g. glutamate, orotate and trigonelline) for SLC22A7 enabling transport systems for the identification of modulators.

### SUMMARY OF THE INVENTION

The present invention is directed to the identification of substrates for SLC22A7 transporter and therapeutic uses thereof. Hence, in one embodiment the present invention relates to a method for identifying and/or obtaining a compound capable of modulating glutamate transport, comprising contacting a test compound with a system for measuring those transport activity, which system comprises an SLC22A7 polypeptide or a functional fragment thereof, and a substrate for measuring glutamate transport by the system; and detecting an altered level of the those transport activity of the SLC22A7 polypeptide or functional fragment in the presence of the test compound compared to the described transport activity in the absence of the test compound and/or presence of a control. This method is useful to identify and obtain drugs for the treatment of disorders related to glutamate transporter function or the lack of it as well as for determining the toxicity of a given compound, for example whether it blocks the glutamate transporter activity. The impact of drug transporter studies on drug discovery and development is reviewed in Mizuno et al., Pharmacol. Rev. 55 (2003), 425-461.

Furthermore, the present invention relates to the use of a compound capable of modulating glutamate transport activity of the SLC22A7 for the manufacture of a medicament for the treatment and/or prophylaxis of a disease related to glutamate transport or glutamate production or glutamate accumulation. In particular, therapeutic intervention through SLC22A17 is envisaged for the treatment of kidney disease or diseases related to cerebral ischemia.

In a further aspect, the present invention relates to the use of a compound capable of modulating glutamate transport activity or expression of the SLC22A7 so as to reduce the intracellular level of the substrates in a target cell for the manufacture of a medicament for inducing cell death in a target cell, This embodiment is particularly suited for the treatment of malignant diseases, in particular cancer.

In addition, the finding of the SLC22A7 enables diagnostic methods for determining the presence of or a susceptibility to a disease or a disorder the SLC22A7 is involved in, which therefore is also subject of the present invention.

The identification of the substrate specificity of SLC22A7 now also enables the person skilled in the art to prepare functional derivatives of the originally described organic cation transporter polypeptides.

The identification of glutamate as the natural substrate of SLC22A7 enables the development and use of a transport or binding assay to identify modulators of SLC22A7 activity.

### BRIEF DESCRIPTION OF THE DRAWINGS

FIG. 1 shows the nucleotide sequence of human SLC22A7 polynucleotide (SEQ ID NO:1).

FIG. 2 shows the amino acid sequence of human SLC22A7 polypeptide (SEQ ID NO:2).

FIG. 3 shows nucleotide sequence of SEQ ID NO:3

FIG. 4 shows nucleotide sequence of SEQ ID NO:4

FIG. 5 shows nucleotide sequence of SEQ ID NO:5

FIG. 6 shows nucleotide sequence of SEQ ID NO:6

FIG. 7 shows nucleotide sequence of SEQ ID NO:7

FIG. 8 shows nucleotide sequence of SEQ ID NO:8

FIG. 9 shows nucleotide sequence of SEQ ID NO:9

FIG. 10 shows nucleotide sequence of SEQ ID NO:10

FIG. 11 shows nucleotide sequence of SEQ ID NO:11

FIG. 12 shows nucleotide sequence of SEQ ID NO:12

FIG. 13 shows nucleotide sequence of SEQ ID NO:13

FIG. 14 shows: OAT2 transports trigonelline into 293 cells. The trigonelline content of cell lysates was determined by LC-MS/MS. Expression of OAT2 in 293 cells increases trigonelline content. 293 cells transfected with pEBTetD plasmids for the expression of SLC22 family carriers as indicated were grown in dishes and incubated overnight with (expression on) or without (off) doxycyclin in standard culture medium. Cells were washed with uptake buffer, incubated (1 min, 37° C.) in uptake buffer with or without 10 μmol/l unlabeled trigonelline, washed, and lysed. Note that OAT2 accumulates trigonelline from the medium during cell culture; thus, acute uptake was insignificant here.

FIG. 15 shows: OAT2 transports trigonelline into 293 cells. Time course of trigonelline uptake. Cells grown in dishes were incubated at 37° C. with 10 μmol/l unlabeled trigonelline in uptake buffer for the indicated periods, washed, and lysed. The function for non-linear regression was y=y0+kin/kout*cout*(1−exp(−kout*x)); parameter y0 allows for trigonelline accumulation from the culture medium. The clearance kin was 0.11±0.01 μl min-1 mg protein-1 for uninduced control cells (open circles) and 5.1±1.4 μl min-1 mg protein-1 for OAT2r-expressing cells filled circles).

FIG. 16 shows: Transport of orotic acid by OAT2. Transfected 293 cells in dishes were incubated (1 min, 37° C.) with 0.1 μmol/l 3H-orotic acid in uptake buffer, washed, and lysed. Cell lysates were analyzed by liquid scintillation counting.

FIG. 17 shows: Transport of orotic acid by OAT2. Time course of uptake of unlabeled orotic acid. See legend to FIG. 15 for basic information. kout was 0.09±0.01 min-1 (expression on) and 0.12±0.03 min-1 (off).

FIG. 18 shows: Transport of orotic acid by OAT2. Saturation of OAT2r-mediated uptake of 3H-orotic acid. An uptake period of 1 min was chosen to approximate initial rates of transport. Non-specific uptake into control cells (expression off) increased linearly with orotic acid concentration, slope=0.56 μl min-1 mg protein-1. It was subtracted from uptake into OAT2r-expressing cells to yield the specific uptake shown. Vmax=13.2±0.4 nmol min-1 mg protein-1. Inset: Eadie-Scatchard transformation.

FIG. 19 shows: Trans-stimulation of efflux of 3H-orotic acid via OAT2r by potential substrates. OAT2r-expressing cells in dishes were incubated (1 h, 37° C.) with 100 μmol/l 3H-orotic acid in uptake buffer, washed twice with ice-cold uptake buffer, and then incubated for 1 min with uptake buffer (control) or 1 mmol/l of the indicated compounds in uptake buffer (1 ml). Finally, uptake buffer (0.7 ml) was collected and analyzed by liquid scintillation counting for released 3H-orotic acid. Directly after washing, OAT2r-expressing cells contained radiolabel corresponding to 56±3 nmol/mg protein orotic acid; uninduced control cells contained 3.5±0.1 nmol/mg protein only (n=4).

FIG. 20 shows: Transport of glutamate by OAT2r. Uptake of 3H-glutamate. Transfected 293 cells in dishes were incubated (1 min, 37° C.) with 0.1 μmol/l 3H-glutamate in uptake buffer (control) or sodium-free buffer without or with 1 mmol/l aspartate as indicated, washed, and lysed. Cell lysates were analyzed by liquid scintillation counting. Sodium-free buffer contained N-methyl-D-glucosamine hydrochloride instead of sodium chloride; pH was adjusted with TRIS instead of NaOH. Immediately before uptake measurement in sodium-free buffer, cells were washed twice with this buffer (heated to 37° C.). In paired dishes, uptake of 3H-orotate was 9.9±0.9 (expression on) and 0.8±0.1 μmol min-1 mg protein-1 (off) in control buffer, and 7.5±0.8 (on) and 0.6±0.1 μmol min-1 mg protein-1 (off) in sodium-free buffer (n=3).

FIG. 21 shows: Transport of glutamate by OAT2r. Efflux of glutamate. Transfected cells in dishes with or without expression of OAT2r were washed with uptake buffer (37° C.), and then incubated for 10 min with uptake buffer (control) or 1 mmol/l of the indicated compounds in uptake buffer (1 ml). Uptake buffer (0.7 ml) was collected and analyzed for released glutamate by LC-MS/MS. A unrelated SLCO5 family transporter was used as additional control.

FIG. 22 shows: Saturation of OAT2h-mediated uptake of 3H-glutamate in sodium-free buffer. An uptake period of 1 min was chosen to approximate initial rates of transport. Uptake into control cells (expression off) was subtracted from uptake into OAT2h-expressing cells to yield the specific uptake shown. Vmax=36±4 nmol min-1 mg protein-1. Inset: Eadie-Scatchard transformation.

FIG. 23 shows: Verification of plasma membrane targeting of OAT2h mutant E441Q by fluorescence microscopy. Transfected 293 cells, grown on poly-L-ornithine-precoated coverslips, were treated for 20 h with doxycycline to turn on expression of eGFP fusion protein, washed with PBS, and then placed on a slide. eGFP was visualized with an Olympus FV1000 IX81 confocal laser scanning microscope (Olympus, Hamburg, Germany) at 488 nm (excitation) and 510 nm (emission).

FIG. 24 shows: Transport of 3H-orotic acid and 3H-glutamate by OAT2h wild-type and mutant E441Q. 293 cells transfected for expression of eGFP chimera of OAT2h wild-type or OAT2hE441Q in dishes were incubated (1 min, 37° C.) with 0.1 μmol/l radiolabeled solute in uptake buffer, washed, and lysed. Cell lysates were analyzed by liquid scintillation counting.

FIG. 25 shows: Localization of OAT2. Tissue distribution of OAT2h analyzed by real-time PCR. Results are given relative to the mRNA level of liver. The following tissues or cells had a signal<0.1%: cerebellum, brain, ovary, 293 cells, spleen, prostate, skin, heart, pancreas, leukocytes (peripheral), skeletal muscle, bone marrow, lung, and placenta.

FIG. 26 shows: Localization of OAT2. Hepatic zonation of OAT2r analyzed by laser capture microdissection followed by real-time PCR. Periportal and pericentral tissue discs were isolated from male rat liver slices; random areas on the same sections were used as control (“total liver”). Results for each mRNA species were scaled to the material with highest content (=100%). Note that OAT2 results were confirmed with a second amplicon. Similar results were obtained with 2 further male rats.

FIG. 27 shows: Transport of glutamate by OAT2r. Uptake of 3H-glutamate. Transfected 293 cells in dishes were incubated (1 min, 37° C.) with 0.1 μmol/l 3H-glutamate in uptake buffer (control) or sodium-free buffer without or with 1 mmol/l aspartate as indicated, washed, and lysed. Cell lysates were analyzed by liquid scintillation counting. Sodium-free buffer contained N-methyl-D-glucosamine hydrochloride instead of sodium chloride; pH was adjusted with TRIS instead of NaOH. Immediately before uptake measurement in sodium-free buffer, cells were washed twice with this buffer (heated to 37° C.). In paired dishes, uptake of 3H-orotate was 9.9±0.9 (expression on) and 0.8±0.1 μmol min-1 mg protein-1 (off) in control buffer, and 7.5±0.8 (on) and 0.6±0.1 μmol min-1 mg protein-1 (off) in sodium-free buffer (n=3).

FIG. 28 shows: Transport of glutamate by OAT2r. Efflux of glutamate. Transfected cells in dishes with or without expression of OAT2r were washed with uptake buffer (37° C.), and then incubated for 10 min with uptake buffer (control) or 1 mmol/l of the indicated compounds in uptake buffer (1 ml). Uptake buffer (0.7 ml) was collected and analyzed for released glutamate by LC-MS/MS. A unrelated SLCO5 family transporter was used as additional control.

FIG. 29 shows: Difference images of cell lysates. 293 cells stably transfected with pEBTetD/OAT2r or pEBTetD/OAT2h splice variant were cultivated for 20 h in the presence (to express the transporter) or absence (control) of 1 μg/ml doxycycline in growth medium. 293 cells do not natively express OAT2h (see legend to FIG. 6A). Cells were directly washed with ice-cold uptake buffer and lysed with methanol. Lysates were analyzed by full scan LC-MS (HILIC column, positive ionization mode, scan time 2 s, m/z-range 75-350).

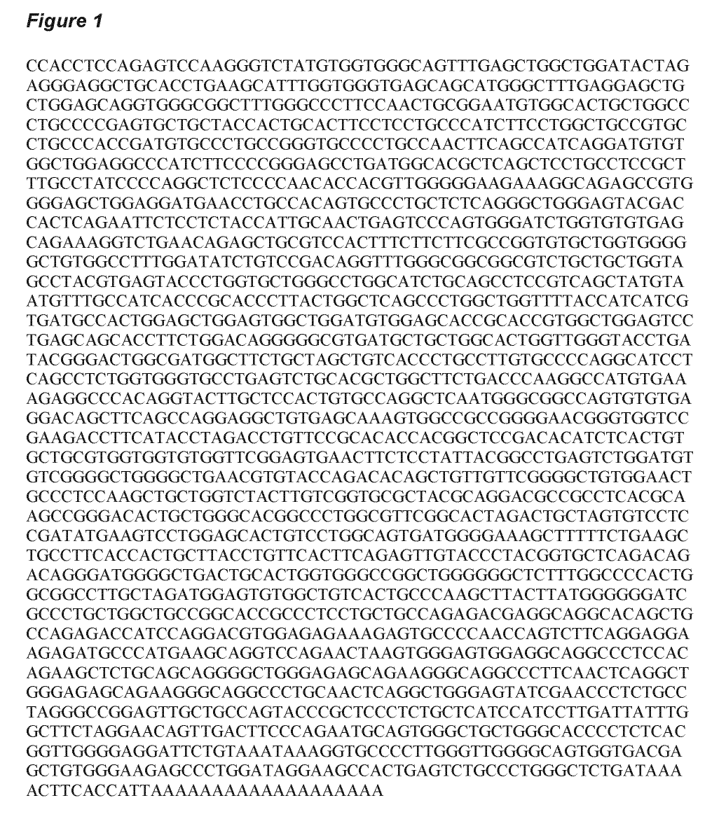

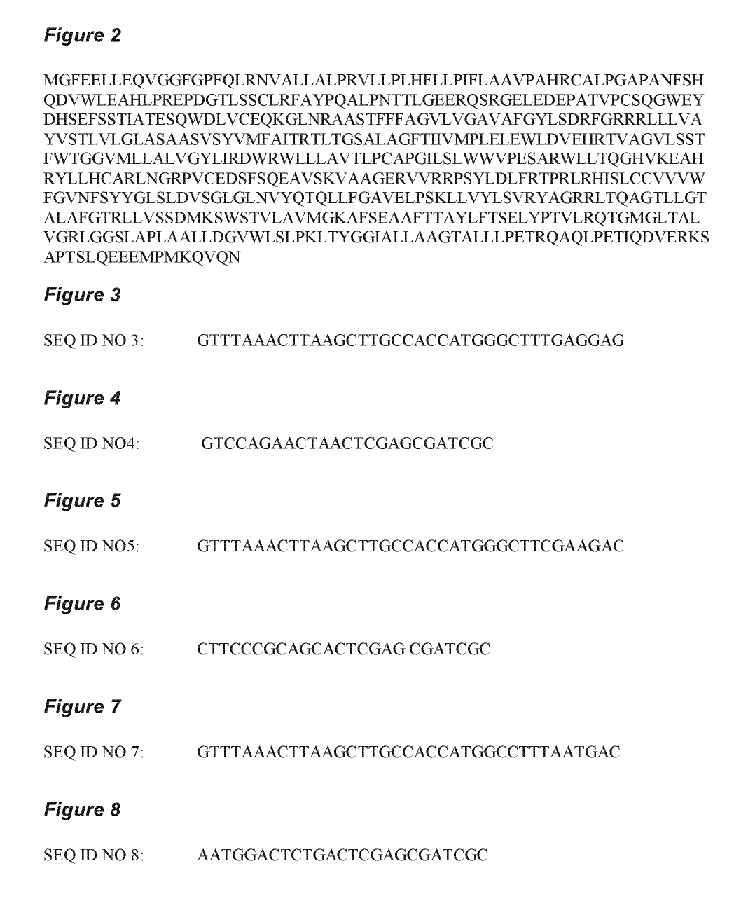

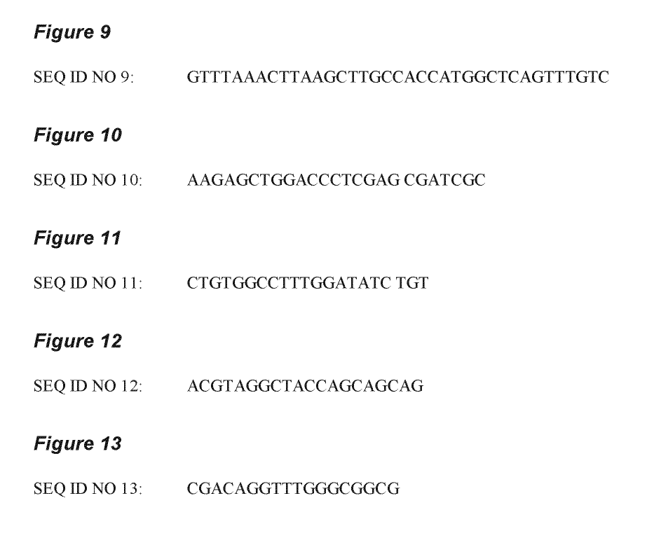

### DETAILED DESCRIPTION OF THE INVENTION

The present invention generally relates to the SLC22A7 transporter and to various uses thereof, for example in therapeutic and diagnostic applications as well as research tool.

### Definitions

“Active”, with respect to a SLC22A7 polypeptide, refers to those forms, fragments, or domains of a SLC22A7 polypeptide which retain the biological and/or antigenic activity of a SLC22A7 polypeptide.

“Naturally occurring SLC22A7 polypeptide” refers to a polypeptide produced by cells which have not been genetically engineered and specifically contemplates various polypeptides arising from post-translational modifications of the polypeptide including but not limited to acetylation, carboxylation, glycosylation, phosphorylation, lipidation and acylation.

“Derivative” refers to polypeptides which have been chemically modified by techniques such as ubiquitination, labeling (see above), pegylation (derivatization with polyethylene glycol), and chemical insertion or substitution of amino acids such as ornithine which do not normally occur in human proteins.

“Conservative amino acid substitutions” result from replacing one amino acid with another having similar structural and/or chemical properties, such as the replacement of a leucine with an isoleucine or valine, an aspartate with a glutamate, or a threonine with a serine.

“Insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

A “signal sequence” or “leader sequence” can be used, when desired, to direct the polypeptide through a membrane of a cell. Such a sequence may be naturally present on the polypeptides of the present invention or provided from heterologous sources by recombinant DNA techniques.

An “oligopeptide” is a short stretch of amino acid residues and may be expressed from an oligonucleotide. Oligopeptides comprise a stretch of amino acid residues of at least 3, 5, 10 amino acids and at most 10, 15, 25 amino acids, typically of at least 9 to 13 amino acids, and of sufficient length to display biological and/or antigenic activity.

“Inhibitor” is any substance which retards or prevents a chemical or physiological reaction or response. Common inhibitors include but are not limited to antisense molecules, antibodies, and antagonists. The term antagonist can be used interchangeably.

“Activator” is any substance which e.g. enhances, stimulates or activates a chemical or physiological reaction or response, e.g. a transport activity. The term agonist can be used interchangeably.

### SLC22A7 Expression

### SLC22A7 Fusion Proteins

Fusion proteins are useful for generating antibodies against SLC22A7 polypeptides and for use in various assay systems. For example, fusion proteins can be used to identify proteins which interact with portions of SLC22A7 polypeptides. Protein affinity chromatography or library-based assays for protein-protein interactions, such as the yeast two-hybrid or phage display systems, can be used for this purpose. Such methods are well known in the art and also can be used as drug screens.

A SLC22A7 fusion protein comprises two polypeptide segments fused together by means of a peptide bond. The first polypeptide segment can comprise at least 54, 75, 100, 125, 139, 150, 175, 200, 225, 250, or 275 contiguous amino acids of SEQ ID NO: 2 or of a biologically active variant, such as those described above. The first polypeptide segment also can comprise full-length SLC22A7.

The second polypeptide segment can be a full-length protein or a protein fragment. Proteins commonly used in fusion protein construction include, but are not limited to β galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, herpes simplex virus (HSV) BP16 protein fusions and G-protein fusions (for example G(alpha)16, Gs, Gi). A fusion protein also can be engineered to contain a cleavage site located adjacent to the SLC22A7.

### Preparation of Polynucleotides

A naturally occurring SLC22A7 polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated SLC22A7 polynucleotides. For example, restriction enzymes and probes can be used to isolate polynucleotide fragments which comprise SLC22A7 nucleotide sequences. Isolated polynucleotides are in preparations which are free or at least 70, 80, or 90% free of other molecules.

SLC22A7 cDNA molecules can be made with standard molecular biology techniques, using SLC22A7 mRNA as a template. SLC22A7 cDNA molecules can thereafter be replicated using molecular biology techniques known in the art. An amplification technique, such as PCR, can be used to obtain additional copies of polynucleotides of the invention, using either human genomic DNA or cDNA as a template.

Alternatively, synthetic chemistry techniques can be used to synthesizes SLC22A7 polynucleotides. The degeneracy of the genetic code allows alternate nucleotide sequences to be synthesized which will encode SLC22A7 having, for example, an amino acid sequence shown in SEQ ID NO: 2 or a biologically active variant thereof.

### Obtaining Polypeptides

SLC22A7 can be obtained, for example, by purification from human cells, by expression of SLC22A7 polynucleotides, or by direct chemical synthesis.

### Protein Purification

SLC22A7 can be purified from any human cell which expresses the receptor, including those which have been transfected with expression constructs which express SLC22A7. A purified SLC22A7 is separated from other compounds which normally associate with SLC22A7 in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

### Expression of SLC22A7 Polynucleotides

To express SLC22A7, SLC22A7 polynucleotides can be inserted into an expression vector which contains the necessary elements for the transcription and translation of the inserted coding sequence. Methods which are well known to those skilled in the art can be used to construct expression vectors containing sequences encoding SLC22A7 and appropriate transcriptional and translational control elements. These methods include in vitro recombinant DNA techniques, synthetic techniques, and in vivo genetic recombination.

A variety of expression vector/host systems can be utilized to contain and express sequences encoding SLC22A7. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding SLC22A7, vectors based on SV40 or EBV can be used with an appropriate selectable marker.

### Bacterial and Yeast Expression Systems

In bacterial systems, a number of expression vectors can be selected. For example, when a large quantity of SLC22A7 is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding SLC22A7 can be ligated into the vector in frame with sequences for the amino-terminal Met and the subsequent 7 residues of β-galactosidase so that a hybrid protein is produced. pIN vectors or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

### Plant and Insect Expression Systems

If plant expression vectors are used, the expression of sequences encoding SLC22A7 can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters can be used. These constructs can be introduced into plant cells by direct DNA transformation or by pathogen-mediated transfection.

An insect system also can be used to express SLC22A7. For example, in one such system Autographa californica nuclear polyhedrosis virus (AcNPV) is used as a vector to express foreign genes in Spodoptera frugiperda cells or in Trichoplusia larvae. Sequences encoding SLC22A7 can be cloned into a non-essential region of the virus, such as the polyhedrin gene, and placed under control of the polyhedrin promoter. Successful insertion of SLC22A7 will render the polyhedrin gene inactive and produce recombinant virus lacking coat protein. The recombinant viruses can then be used to infect S. frugiperda cells or Trichoplusia larvae in which SLC22A7 can be expressed.

### Mammalian Expression Systems

A number of viral-based expression systems can be used to express SLC22A7 in mammalian host cells. For example, if an adenovirus is used as an expression vector, sequences encoding SLC22A7 can be ligated into an adenovirus transcription/translation complex comprising the late promoter and tripartite leader sequence. Insertion in a non-essential E1 or E3 region of the viral genome can be used to obtain a viable virus which is capable of expressing SLC22A7 in infected host cells [Engelhard, 1994)]. If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles). Specific initiation signals also can be used to achieve more efficient translation of sequences encoding SLC22A7. Such signals include the ATG initiation codon and adjacent sequences. In cases where sequences encoding SLC22A7, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic.

### Host Cells

A host cell strain can be chosen for its ability to modulate the expression of the inserted sequences or to process the expressed SLC22A7 in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express SLC22A7 can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced SLC22A7 sequences. Resistant clones of stably transformed cells can be proliferated using tissue culture techniques appropriate to the cell type. Any number of selection systems can be used to recover transformed cell lines. These include, but are not limited to, the herpes simplex virus thymidine kinase [Logan, (1984)] and adenine phosphoribosyltransferase [Wigler, (1977)] genes which can be employed in tk⁻ or aprt⁻ cells, respectively. Also, antimetabolite, antibiotic, or herbicide resistance can be used as the basis for selection. For example, dhfr confers resistance to methotrexate [Lowy, (1980)], npt confers resistance to the aminoglycosides, neomycin and G-418 [Wigler, (1980)], and als and pat confer resistance to chlorsulfuron and phosphinotricin acetyltransferase, respectively [Colbere-Garapin, 1981]. Additional selectable genes have been described. For example, trpB allows cells to utilize indole in place of tryptophan, or hisD, which allows cells to utilize histinol in place of histidine. Visible markers such as anthocyanins, β-glucuronidase and its substrate GUS, and luciferase and its substrate luciferin, can be used to identify transformants and to quantify the amount of transient or stable protein expression attributable to a specific vector system

### Identification of SLC22A7 Substrates

OAT2 (human gene symbol SLC22A7) is a member of the SLC22 family of transport proteins. The sequences are described under gene ID 10864. In rat, the principal site of expression of OAT2 is the sinusoidal membrane domain of hepatocytes. The particular physiological function of OAT2 has been unresolved so far. We have used the strategy of LC-MS difference shading to search for specific and cross-species substrates of OAT2. Heterologous expression of human and rat OAT2 in human embryonic kidney 293 cells stimulated accumulation of the zwitterion trigonelline; by contrast, expression of OAT1 (SLC22A6) and SLC22AI 3 was without effect. With trigonelline as substrate lead, orotic acid was identified as excellent and specific substrate of OAT2 from rat (clearance=106 μl min-1 mg protein-1) and human (46 μl min-1 mg protein-1). The force driving uptake of orotic acid was identified as glutamate antiport. Efficient transport of glutamate by OAT2 was directly demonstrated by uptake of 3H-glutamate. Because of high (>2 mmol/l) intracellular glutamate in hepatocytes and 293 cells, OAT2 operates as glutamate efflux transporter. This was established by LC-MS analysis: expression of OAT2 markedly increased release of glutamate from cells—even without extracellular exchange substrate. Orotic acid strongly trans-stimulated efflux of glutamate. After laser capture microdissection of rat liver slices, OAT2 mRNA was detected equally in periportal and pericentral regions.

Based on functional studies, we have surprisingly identified that OAT2 (SLC22A7) physiologically functions as glutamate efflux transporter.

### Use of Modulators of SLC22A7

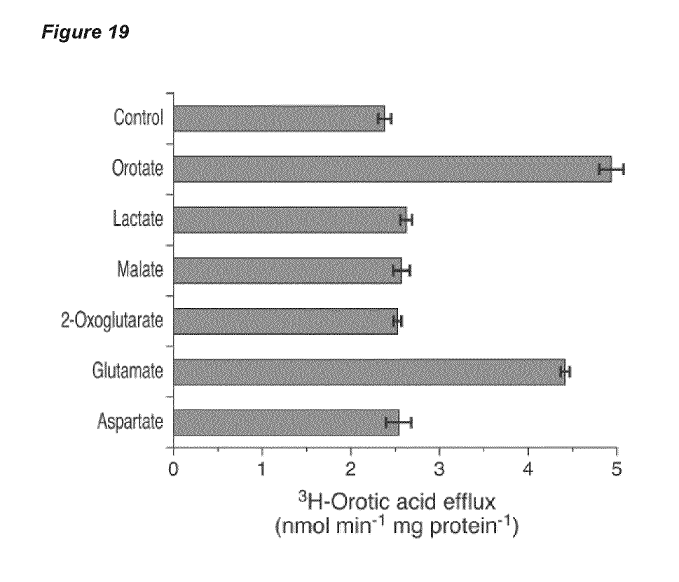

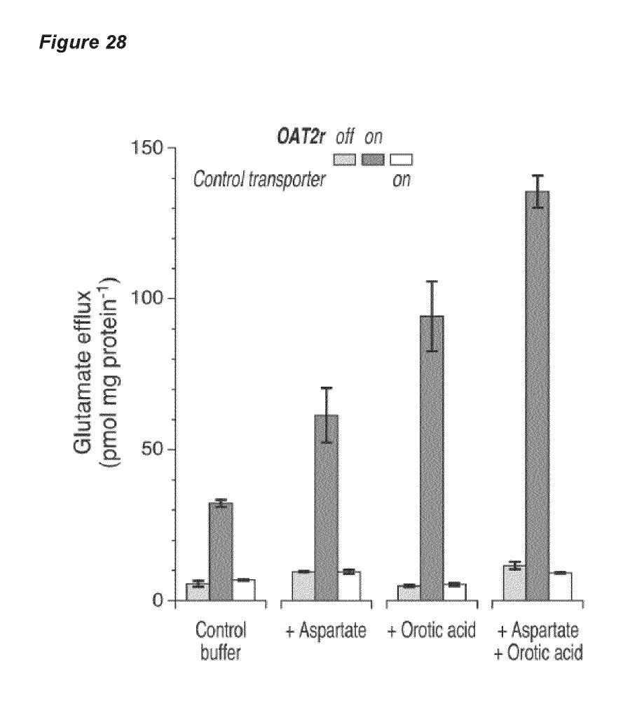

Our results demonstrate that OAT2 (gene symbol SLC22A7) can catalyze both uptake and efflux of glutamate. However, since glutamate is the most abundant intracellular amino acid (reported concentrations, including hepatocytes, range from 2 to 20 mmol/l (Newsholme et al. Cell Biochem Funct 2003; 21:1-9.)), OAT2 effectively operates as glutamate efflux carrier. Notably, even without any extracellular exchange substrate, the carrier releases glutamate from cells (see FIG. 28). The moderate affinity (Km OAT2h=1.1 mmol/l; measured as uptake) reflects that the purpose of OAT2 is not to collect glutamate from extracellular space, but to provide an exit passage for intracellular cytosolic glutamate. As the intracellular glutamate concentration is higher than the Km, strong efflux is generated nevertheless. The benefit of lower-than-possible glutamate transport efficiency (range: 24 to 70 μl min-1 mg protein-1) is supreme substrate selectivity: OAT2 rejects other abundant intracellular anions like lactate, malate, 2-oxoglutarate, even the extremely similar aspartate (FIG. 19). By striking contrast, the high affinity, sodium-driven EAAT-type glutamate uptake transporters all move aspartate and glutamate equally well (23, 24). Thus, our results suggest that the general physiological purpose of OAT2 is to release glutamate from cells. Since the related carriers OAT1, OAT3, and SLC22AI 3 do not transport glutamate, but 2-oxoglutarate, OAT2 is unique within the SLC22 family.

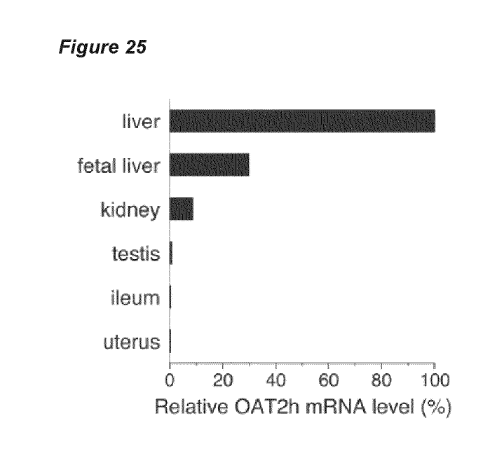

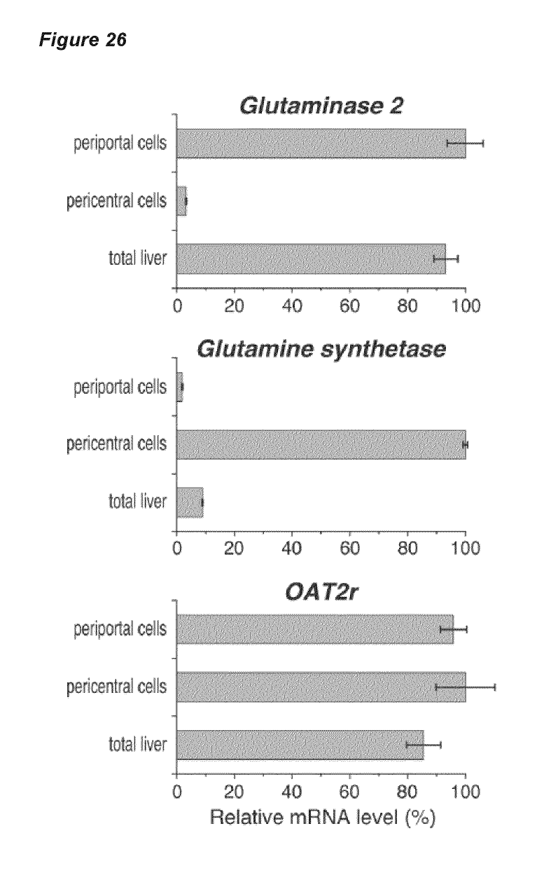

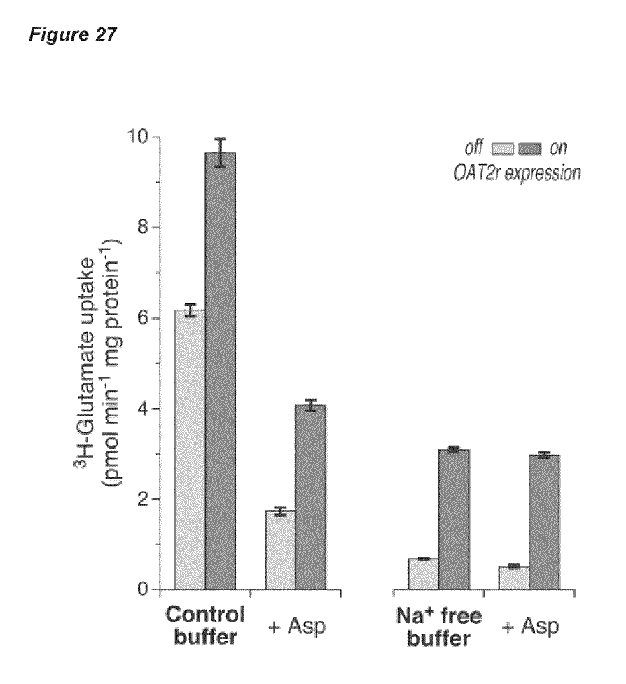

Our real-time PCR data indicate that OAT2 from human is primarily expressed in liver, kidney and CNS (FIG. 25). Similar results were obtained for rat. Our LCM results (FIG. 26) suggest that expression of OAT2r is homogenous across hepatocytes, confined neither to periportal (marker=glutaminase 2) nor to pericentral regions (glutamine synthetase).

If the assignment of OAT2 as hepatic glutamate efflux transporter is correct, then there should be a corresponding phenotype. Indeed, release of glutamate from liver into blood has been reported: 1.) In rats fed a protein-free diet, glutamate was released at 64 μmol/l; this was calculated as the plasma concentration difference between portal vein and hepatic vein. 2.) Isolated perfused rat liver continuously (observation time=100 min) released glutamate at a rate of 40-60 nmol min-1 g-1; this export was sodium-independent. 3.) Under diets either slightly deficient (11% casein) or moderately surfeit (22% casein) in protein, glutamate and glutamine were markedly released by rat liver. Collectively, these observations can now be explained by OAT2 activity and thus reinforce the hypothesis that OAT2 functions as hepatic glutamate efflux transporter. Moreover, with isolated perfused rat liver both periportal and pericentral release of glutamate was observed; this conforms to our LCM data on OAT2r. The extent of hepatic release of glutamate was documented further by recent experiments with pigs: in a model of acute liver failure induced by portacaval shunt and hepatic artery ligation, the arterial glutamate concentration was reduced 6-fold 2 h after induction (67 vs. 416 μmol/l).

A marked decrease of plasma glutamate was also measured in patients with liver failure (Clemmesen et al. Gastroenterology 2000; 118:1131-1139; Strauss et al. Gastroenterology 2001; 121:1109-1119; Schmidt et al. Scand J Gastroenterol 2004; 39:974-980.), cirrhosis (Clemmesen et al. Gastroenterology 2000; 118:1131-1139), and acute on chronic liver disease (Clemmesen et al. Gastroenterology 2000; 118:1131-1139; Schmidt et al. Scand J Gastroenterol 2004; 39:974-980.). Moreover, patients who die of septic shock with acute liver dysfunction can be predicted by significantly lowered plasma glutamate concentrations and lowered glutamate/glutamine ratios (Poeze et al. Clin Nutr 2008; 27:523-530.). Thus, the liver—via OAT2 as we can now suggest—maintains glutamate levels in plasma; glutamate absorbed from food normally has no role here, since it is metabolized in the intestine and released primarily as alanine. It follows that plasma glutamate depletion is a feature of liver failure like loss of ureagenesis.

In mammalia, glutamate and glutamine are extraordinary amino acids, since both are constantly released from liver into blood. At the same time, there is uptake of glutamine into periportal hepatocytes (=zone of glutamine hydrolysis and urea production) and uptake of glutamate into pericentral hepatocytes (=zone of glutamine synthesis and release). Glutamine serves as preferred energy fuel for rapidly proliferating cells (e.g. enterocytes of the intestine; lymphocytes) and as substrate for ammoniagenesis; it has the highest plasma concentration (e.g. human artery: 0.57±0.04 mmol/l (31)) of all protein monomers. By contrast, there is 10-20 times less glutamate in human plasma (e.g. human artery: 57±5 μM; hepatic vein: 134±14 μM). It is assumed that glutamate is only utilized intracellularly. Since glutamate is highly hydrophilic, its distribution into organs is entirely controlled by membraneinserted transport proteins like the sodium-driven EAATs. Intracellularly, there are several enzymes that can directly use glutamate; an interesting candidate is glutamine synthetase, which converts glutamate and ammonia to glutamine. Since ammonia is toxic at elevated levels, it is feeded into the urea alias ornithine cycle in the liver in most mammals. The most important alternative detoxification pathway is glutamine synthesis followed by release of non-toxic glutamine into blood and glutamine hydrolysis and ammonia secretion in the kidney (Meijer et al. Physiol Rev 1990; 70:701-748.).

Glutamine synthetase has high affinity for ammonia, allowing the removal of low ammonia concentrations. Important sites of glutamine synthesis from blood ammonia are pericentral hepatocytes (Watford M. J Nutr 2000; 130:983 S-987S.), muscle (Olde et al. Metab Brain Dis 2009; 24:169-181.), and lung (Souba et al. JPEN J Parenter Enteral Nutr 1990; 14:68 S-70S.), but probably not brain. Another important site of ammonia scavenging may be platelets, which express glutamine synthetase and EAAT2.

Interestingly, thrombin activation of platelets increases glutamate uptake up to 9-fold by translocation of EAAT2 to the outer membrane (Hoogland et al. Neurochem Int 2005; 47:499-506.). The role of glutamate and glutamate transporters for platelets is still unclear. It can be speculated, however, that plasma concentrations of glutamate are high enough to sustain efficient local ammonia detoxification. On the whole, there is some evidence to suggest that plasma glutamate is a key substrate for ammonia detoxification via intracellular glutamine synthetase.

Transport of glutamate is the primary, but not the only catalytic function of OAT2. The steep outwardly directed glutamate gradient (see above) provides a powerful driving force for the uptake of selected solutes. We have discovered orotic acid as an outstanding and specific substrate of OAT2. Orotic acid is an intermediate in pyrimidine biosynthesis and a natural dietary constituent (e.g. in dairy products and root vegetables). Orotic acid transport proteins have been identified in bacteria, but not in mammalia. Rat liver absorbs orotic acid rapidly.

### SLC22A7 Modulators

One option is the development of a specific efflux blocker, which can markedly lower plasma glutamate. This could be useful to alleviate glutamate exotoxicity in the CNS as follows: glutamate is the major excitatory neurotransmitter in the vertebrate CNS, but excessive extracellular glutamate is highly toxic to neurons. Some of the glutamate is cleared by brain-to-blood efflux; a reduction of plasma glutamate promotes this efflux. Thus, reduction of plasma glutamate may have important implications in the treatment of acute brain conditions, including closed head injury and stroke. A reduction can be achieved by infusion of substrates of glutamate-oxaloacetate transaminase or glutamate-pyruvate transaminase. However, inhibition of OAT2 is a superior alternative.

Another option for therapeutic intervention is the increase of glutamate efflux via OAT2. This can compensate the loss of plasma glutamate described above in liver failure. Indeed, infusion of glutamate has been successfully used in coma patients with liver failure. Administration of L-ornithine plus L-aspartate also aims to increase glutamate availability for glutamine synthesis to lower ammonia e.g. in hepatic encephalopathy.

A second option is the development of a specific efflux activator or enhancer, which can increase the glutamate efflux from cells. SLC22A7 transport enhancer or activators main be useful for the treatment of kidney diseases.

### SLC22A7 is a Trigonelline Transporter

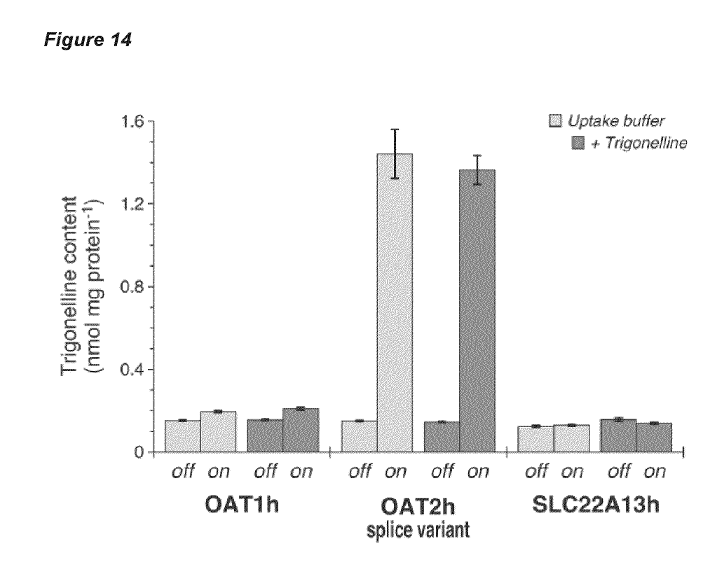

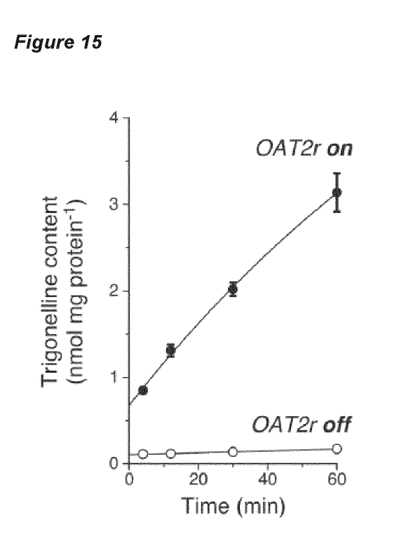

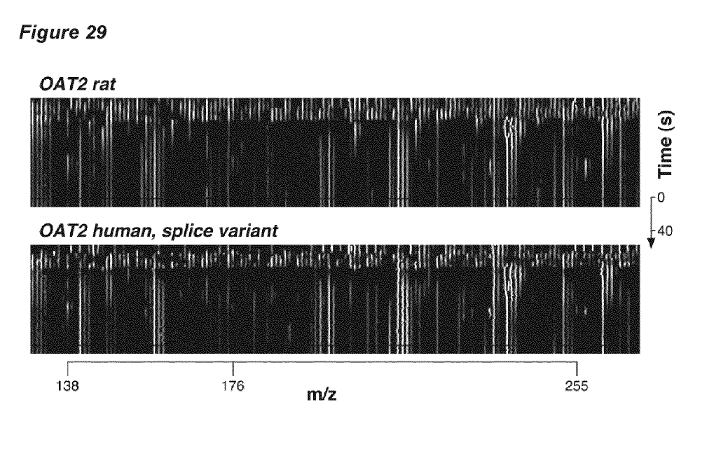

A substrate search by LC-MS difference shading was performed with 293 cells stably transfected to express OAT2 from rat. A strong red signal was reproducibly found at m/z=138 (FIG. 29). An echo at 176 suggested the presence of a carboxylate moiety, complexed with K+ instead of H+. Fragmentation analysis by LC-MS/MS produced major peaks at 94, 92, 78, and 65. The compound was identified as trigonelline (alias 1-methylnicotinic acid or nicotinic acid N-methylbetaine) by comparison with the product ion spectrum of a commercial sample. The signal at m/z=255 was identified as complex of trigonelline and glycine betaine. Expression of OAT2h or OAT2h splice variant E131_W132insSQ also increased trigonelline. In good agreement with the above results, after 24 h incubation with 100 μmol/l trigonelline in uptake buffer, trigonelline was markedly increased, measured by LC-MS/MS, in lysates of cells expressing OAT2r (f=8.1) or OAT2h splice variant (f=3.9) versus control cells. By contrast, after 24 h incubation with 100 μmol/l nicotinic acid or nicotinamide in uptake buffer, there was no significant increase of trigonelline, nicotinic acid, or nicotinamide. To verify that trigonelline is directly transported by OAT2 and not just increased indirectly, initial rates of uptake (1 min, 37° C.) of trigonelline (10 μmol/l) were determined by LC-MS/MS. Initial endogenous trigonelline content was determined with matching cells that were incubated merely in uptake buffer; this background was largely reduced by replacement of standard cell culture medium (which by our measurement contained 8 μmol/l trigonelline) with uptake buffer in the transporter expression period (about 20 h). Under these conditions, the carrier-mediated clearance of trigonelline was 121±5 μl min-1 mg protein-1 for OAT2h, but only 4.8±0.5 μl min-1 mg protein-1 for OAT2r, and 2.5±0.9 μl min-1 mg protein-1 for OAT2h splice variant. Accumulation of trigonelline upon expression of OAT2 is carrier-specific, since the related OAT1 and SLC22A13 were without effect (FIG. 14). In analogous 1 min uptake experiments (10 μmol/l), no transport by OAT2r was observed with nicotinic acid, nicotinamide, 1-methylnicotinamide, and nicotinic acid mononucleotide. However, nicotinic acid riboside was transported slightly more efficiently than trigonelline. A time course after cell culture in full medium (FIG. 15) further corroborated the notion of direct uptake, since there was no lag in trigonelline accumulation. Expression of OAT2r increased the clearance by a factor of almost 50.

Orotic Acid is an Excellent and Specific Substrate of OAT2 from Rat and Human

With trigonelline as substrate lead, compounds wholly or partly related in structure were analyzed for transport by OAT2 from rat. After 24 h incubation (each compound at 100 μmol/l), trigonelline was on average (n=3) increased in OAT2r cell lysates by a factor of 4.7 versus control cells, whereas accumulation of 2-aminobenzoic acid (f=1.1), 3-aminobenzoic acid (1.4), 4-aminobenzoic acid (1.1), L-carnitine (1.0), 3-(diethylamino)propionic acid (1.1), ectoine (1.2), gabapentin (1.2), glycine betaine (1.0), riboflavin (1.2), and stachydrine (1.2) was virtually unaffected by transporter expression. Quinolinic acid was increased by a factor of 2.7, but there was virtually no difference in lysate content between OAT2r and control cells up to 1 h incubation time; it was concluded that quinolinic acid is no substrate of OAT2, but increased indirectly by an unkown pathway.

In 1 min uptake experiments (0.1 μmol/l radiotracer or 10 μmol/l otherwise), no transport by OAT2r was observed with 2-aminobenzoic acid, 3-aminobenzoic acid, 4-aminobenzoic acid, 5-aminolevulinic acid, creatine, guanidinoacetic acid, L-proline, riboflavin, stachydrine, and thiamine.

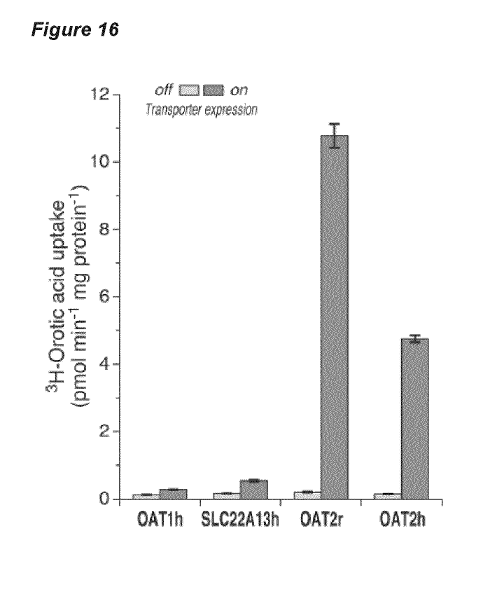

Surprisingly, in 1 min uptake experiments with 0.1 μmol/l radiotracer (FIG. 16), orotic acid was transported with supreme efficiency by OAT2r (clearance=106±4 μl min-1 mg protein-1). Vigorous transport was also observed with OAT2 from human (46±1 μl min-1 mg protein-1). By contrast, transport by SLC22A13h (3.8±0.3 μl min-1 mg protein-1), OAT1h (1.6±0.1 μl min-1 mg protein-1), and OAT3h (0.7±0.1 μl min-1 mg protein-1) was very low.

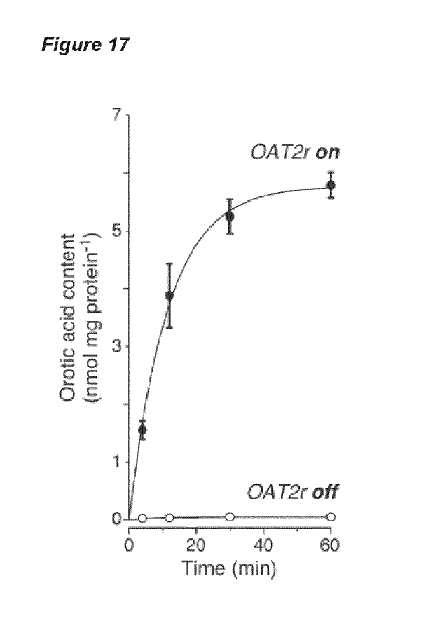

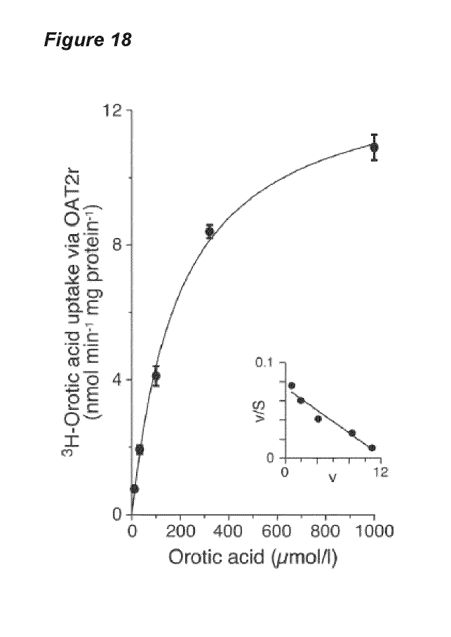

A time course (FIG. 17) with 10 μmol/l unlabeled orotic acid in uptake buffer revealed that OAT2r elevates intracellular orotic acid, selectively measured by LC-MS/MS, to a plateau of 5.8 nmol/mg protein after 60 min; this corresponds to 870 μmol/l (calculated with an intracellular water space of 6.7 μl/mg protein). By contrast, cells lacking the transporter reached only 0.05 nmol/mg protein after 60 min. The clearance (kin) was increased from 0.58±0.12 to 50.1±6.4 μl min-1 mg protein-1 by expression of OAT2r, i.e. by a factor of 86. In an analogous experiment with 3H-orotic acid, radioactivity was still on the rise after 60 min, probably because of intracellular metabolism of 3H-orotic acid. However, kinetic values for OAT2r expression were similar. Thus, intracellular metabolism does not drive uptake of orotic acid. Saturation analysis of OAT2r-mediated uptake of 3H-orotic acid (FIG. 18) revealed a Km of 200 (95% confidence interval: 160-240) μmol/l, which for a transport protein indicates moderate affinity. To further elaborate the specificity of OAT2r, compounds related in structure to orotic acid were assayed. In 1 min uptake experiments with unlabeled compounds (10 μmol/l) and LC-MS/MS quantitation, no transport was observed with L-dihydroorotic acid, kynurenic acid, 5-oxoproline, hydantoin-5-acetic acid, and barbituric acid. Only 5-fluoroorotic acid and isoorotic acid (alias uracil-5-carboxylic acid), which are very similar in structure to orotic acid, were transported efficiently. This indicates stringent substrate selectivity of OAT2r.

### SLC22A7 is a Glutamate Transporter

If OAT2r would simply facilitate diffusion, then the monoanion orotic acid (pKa≈2.4 (15, 16)) should only accumulate, at regular membrane potential and 10 μmol/l extracellular, to roughly 1 16 μmol/l intracellular. Full replacement of sodium chloride in the uptake buffer by choline chloride, lithium chloride, or potassium chloride, or complete omission of magnesium and calcium reduced uptake of orotic acid only slightly, by a factor of 0.75 on average; thus, cotransport of inorganic cations was ruled out.

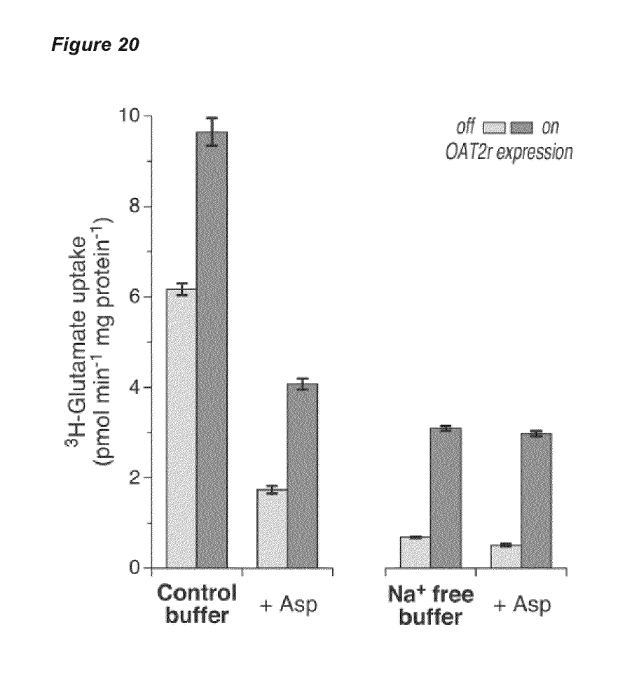

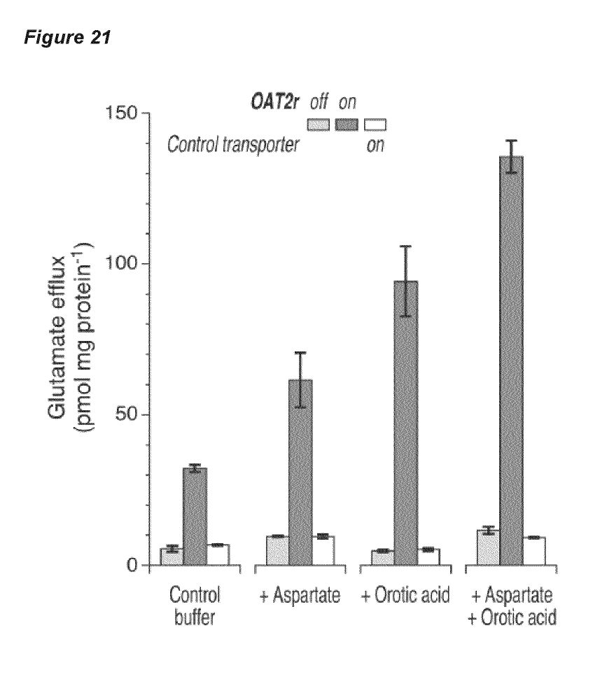

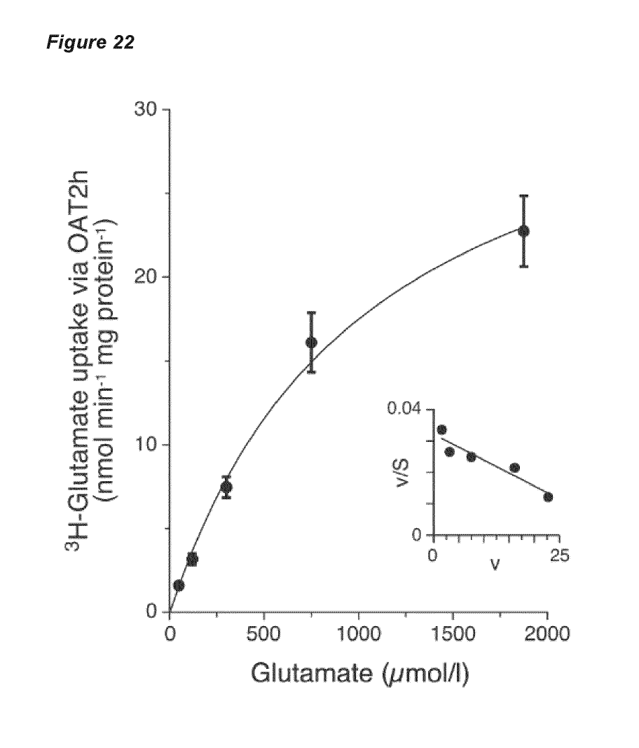

To probe for trans-stimulation, OAT2r-expressing cells were preincubated (1 h) with uptake buffer±100 μmol/l unlabeled orotic acid, washed twice with ice-cold uptake buffer, and then assayed for uptake of 3H-orotic acid (0.1 μmol/l, 1 min) and trigonelline (10 μmol/l, 5 min). Interestingly, preloading with orotic acid reduced uptake of both substrates (f=0.7). This trans-inhibition could mean that 293 cells contain a compound that is expelled faster by OAT2r than the competitor orotic acid. Efflux of 3H-orotic acid from preloaded cells into uptake buffer was measured (1 min) to identify compounds that can drive OAT2r. The assay was validated with unlabeled orotic acid (1 mmol/l) in uptake buffer, which markedly accelerated efflux (“trans-stimulation”) over control (FIG. 19). Among several anionic candidate compounds which are present in liver cells at substantial concentrations (>0.1 mmol/l) (Parrilla et al., Pflugers Arch 1976; 362:49-54; Brosnan et al. Biochem J 1970; 117:91-96.), only glutamate accelerated efflux of 3H-orotic acid (FIG. 19). Transport of glutamate by OAT2r was directly demonstrated by uptake of 3H-glutamate (FIG. 20). For a better signal-to-noise ratio, background uptake (OAT2r expression off), probably caused to a large part by an EAAT-type glutamate transporter endogenous to 293 cells, was reduced strongly with sodium-free uptake buffer or by addition of 1 mmol/l aspartate; the clearance of glutamate by OAT2r was 35±3 μl min-1 mg protein-1 in standard buffer and 24±1 μl min-1 mg protein-1 in sodium-free buffer. OAT2 from human was even more active (70±5 μl min-1 mg protein-1, sodium-free buffer). By striking contrast, OAT1h (2.5±0.4 μl min-1 mg protein-1), OAT3h (0.2±0.6 μl min-1 mg protein-1), and SLC22A13h (0.3±0.6 μl min-1 mg 17 protein-1) generated no or very little uptake of glutamate. Saturation analysis of OAT2h mediated uptake of 3H-glutamate in sodium-free buffer (FIG. 22) revealed a Km of 1.1 (0.6-1.8) mmol/l. OAT2h generated no uptake of 3H-glutamine. In view of high (>2 mmol/l) intracellular glutamate in hepatocytes (Geerts et al. J Histochem Cytochem 1997; 45:1217-1229.), our above results suggest that OAT2 operates as glutamate efflux transporter. To test this hypothesis, 293 cells with or without expression of OAT2r were incubated 10 min in uptake buffer; then, the glutamate content of a buffer sample was determined by LC-MS/MS. Expression of OAT2 markedly increased release of glutamate from the cells—even without extracellular exchange substrate (FIG. 21, control buffer). Addition of 1 mmol/l aspartate blocked the endogenous EAAT-type glutamate uptake transporter and thus revealed even higher glutamate efflux. Addition of 1 mmol/l orotic acid strongly trans-stimulated efflux of glutamate. As consequence of continuous efflux, the intracellular glutamate content was clearly reduced in 293 cells expressing OAT2r (f=0.55±0.11; n=4) or OAT2h (f=0.60±0.09; n=2) versus uninduced control cells.

### Characterization of SLC22A7

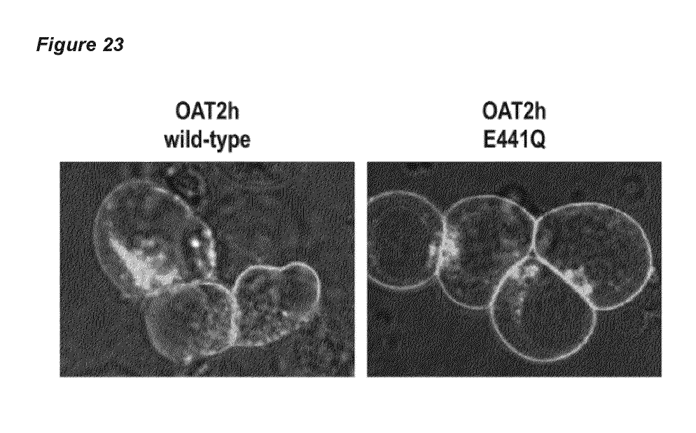

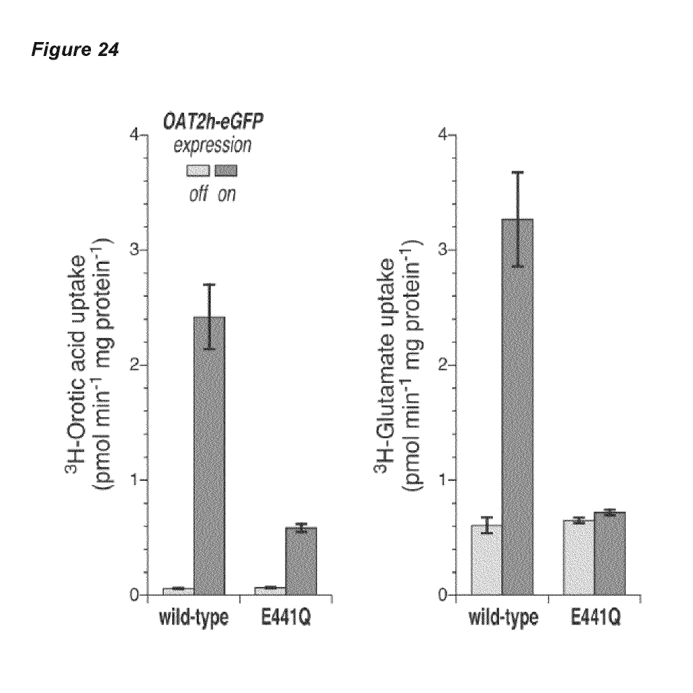

In transmembrane segment 10, the OAT2 orthologues from human, horse, pig, cattle, rabbit, rat, mouse, opossum, and chicken uniformly contain a glutamate (E441 in OAT2h) that is absent in the related carriers OAT1, OAT3, and SLC22A13. Since in other SLC22 family members transmembrane segment 10 is involved in substrate discrimination (Bacher et al. Biochim Biophys Acta 2009; 1788:2594-2602.), the negative charge of the glutamate side chain could specifically attract the ammonium residue of the glutamate substrate. To test this hypothesis, a mutant of OAT2h was created with glutamine at position 441 (E441 Q); eGFP was attached to the C-terminus to verify unaltered membrane trafficking (FIG. 23). With the mutant, uptake of 3H-glutamate was completely abolished (FIG. 24); by contrast, uptake of 3H-orotic acid was much reduced, but still evident. This reduction probably reflects the loss of driving force (=glutamate efflux) rather than reduced orotic acid transport competence. Our results thus suggest that OAT2h mutant E441Q is folded and sorted like the wild-type carrier. It follows that E441 is essential for transport of glutamate.

Regulators as used herein, refer to compounds that affect the activity of a SLC22A7 in vivo and/or in vivo. Regulators can be inhibitors or activators of a SLC22A7 polypeptide and can be compounds that exert their effect on the SLC22A7 activity via the expression, via post-translational modifications or by other means. Activators of SLC22A7 are molecules which, when bound to SLC22A7, increase or prolong the activity of SLC22A7. Activators of SLC22A7 include proteins, nucleic acids, carbohydrates, small molecules, or any other molecule which activate SLC22A7. Inhibitors of SLC22A7 are molecules which, when bound to SLC22A7, decrease the amount or the duration of the activity of SLC22A7. Inhibitors include proteins, nucleic acids, carbohydrates, antibodies, small molecules, or any other molecule which decrease the activity of SLC22A7.

The term “modulate”, as it appears herein, refers to a change in the activity of SLC22A7 polypeptide. For example, modulation may cause an increase or a decrease in protein activity, binding characteristics, or any other biological, functional, or immunological properties of SLC22A7.

As used herein, the terms “specific binding” or “specifically binding” refer to that interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A” the presence of a polypeptide containing the epitope A, or the presence of free unlabeled A, in a reaction containing free labeled A and the antibody will reduce the amount of labeled A that binds to the antibody.

The invention provides methods (also referred to herein as “screening assays”) for identifying compounds which can be used for the treatment of cardiovascular diseases, disorders of the central nervous system, kidney and liver diseases. The methods entail the identification of candidate or test compounds or agents (e.g., peptides, peptidomimetics, small molecules or other molecules) which bind to SLC22A7 and/or have a stimulatory or inhibitory effect on the biological activity of SLC22A7 or its expression and then determining which of these compounds have an effect on symptoms or diseases regarding cardiovascular diseases, disorders of the central nervous system, kidney and liver diseases in an in vivo assay.

Candidate or test compounds or agents which bind to SLC22A7 and/or have a stimulatory or inhibitory effect on the activity or the expression of SLC22A7 are identified either in assays that employ cells which express SLC22A7 on the cell surface (cell-based assays) or in assays with isolated SLC22A7 (cell-free assays). The various assays can employ a variety of variants of SLC22A7 (e.g., full-length SLC22A7, a biologically active fragment of SLC22A7, or a fusion protein which includes all or a portion of SLC22A7). Moreover, SLC22A7 can be derived from any suitable mammalian species (e.g., human SLC22A7, rat SLC22A7 or murine SLC22A7). The assay can be a binding assay entailing direct or indirect measurement of the binding of a test compound or a known SLC22A7 ligand to SLC22A7. The assay can also be an activity assay entailing direct or indirect measurement of the activity of SLC22A7. The assay can also be an expression assay entailing direct or indirect measurement of the expression of SLC22A7 mRNA or SLC22A7 protein. The various screening assays are combined with an in vivo assay entailing measuring the effect of the test compound on the symptoms of cardiovascular diseases, disorders of the central nervous system, kidney and liver diseases.

In one embodiment, the invention provides assays for screening candidate or test compounds which bind to or modulate the activity of a membrane-bound (cell surface expressed) form of SLC22A7. Such assays can employ full-length SLC22A7, a biologically active fragment of SLC22A7, or a fusion protein which includes all or a portion of SLC22A7. As described in greater detail below, the test compound can be obtained by any suitable means, e.g., from conventional compound libraries. Determining the ability of the test compound to bind to a membrane-bound form of SLC22A7 can be accomplished, for example, by coupling the test compound with a radioisotope or enzymatic label such that binding of the test compound to the SLC22A7-expressing cell can be measured by detecting the labeled compound in a complex. For example, the test compound can be labelled with ¹²⁵I, ³⁵S, ¹⁴C, or ³H, either directly or indirectly, and the radioisotope detected by direct counting of radioemmission or by scintillation counting. Alternatively, the test compound can be enzymatically labelled with, for example, horseradish peroxidase, alkaline phosphatase, or luciferase, and the enzymatic label detected by determination of conversion of an appropriate substrate to product.

In a competitive binding format, the assay comprises contacting SLC22A7 expressing cell with one of the identified natural substrates SLC22A7 to form an assay mixture, contacting the assay mixture with a test compound, and determining the ability of the test compound to interact with the SLC22A7 expressing cell, wherein determining the ability of the test compound to interact with the SLC22A7 expressing cell comprises determining the ability of the test compound to preferentially bind the SLC22A7 expressing cell as compared to the identified natural substrates.

In another embodiment, the assay is a cell-based assay comprising contacting a cell expressing a membrane-bound form of SLC22A7 (e.g., full-length SLC22A7, a biologically active fragment of SLC22A7, or a fusion protein which includes all or a portion of SLC22A7) expressed on the cell surface with a test compound and determining the ability of the test compound to modulate (e.g., stimulate or inhibit) the activity of the membrane-bound form of SLC22A7. Determining the ability of the test compound to modulate the activity of the membrane-bound form of SLC22A7 can be accomplished by any method suitable for measuring the activity of SLC22A7, e.g., any method suitable for measuring the activity of a G-protein coupled receptor or other seven-transmembrane receptor (described in greater detail below). The activity of SLC22A7 can be measured in a number of ways. Amongst others, the measures of activity are: alteration in intracellular concentration of trigonelline, glutamate or orotate, and alteration in extracellular concentration of trigonelline, glutamate or orotate. The concentration of the aforementioned substrates can be determined e.g. by using radio-labeled substrates.

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds [Lam, (1997)]. Examples of methods for the synthesis of molecular libraries can be found in the art. Libraries of compounds may be presented in solution or on beads, bacteria, spores, plasmids or phage.

### Production of Altered Polypeptides

As will be understood by those of skill in the art, it may be advantageous to produce SLC22A7 polynucleotides possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences referred to herein can be engineered using methods generally known in the art to alter SLC22A7 polynucleotides for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

### Antibodies

Any type of antibody known in the art can be generated to bind specifically to an epitope of SLC22A7.

“Antibody” as used herein includes intact immunoglobulin molecules, as well as fragments thereof, such as Fab, F(ab′)₂, and Fv, which are capable of binding an epitope of SLC22A7, preferably an epitope comprising the glutamate at position 441. Typically, at least 6, 8, 10, or 12 contiguous amino acids are required to form an epitope. However, epitopes which involve non-contiguous amino acids may require more, e.g., at least 15, 25, or 50 amino acid. An antibody which specifically binds to an epitope of SLC22A7 can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the SLC22A7 immunogen.

Typically, an antibody which specifically binds to SLC22A7 provides a detection signal at least 5-, 10-, or 20-fold higher than a detection signal provided with other proteins when used in an immunochemical assay. Preferably, antibodies which specifically bind to SLC22A7 do not detect other proteins in immunochemical assays and can immunoprecipitate SLC22A7 from solution.

SLC22A7 can be used to immunize a mammal, such as a mouse, rat, rabbit, guinea pig, monkey, or human, to produce polyclonal antibodies. If desired, SLC22A7 can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Canmette-Guerin) and Corynebacterium parvum are especially useful.

Monoclonal antibodies which specifically bind to SLC22A7 can be prepared using any technique which provides for the production of antibody molecules by continuous cell lines in culture. These techniques include, but are not limited to, the hybridoma technique, the human B-cell hybridoma technique, and the EBV-hybridoma technique [Roberge, (1995)].

In addition, techniques developed for the production of “chimeric antibodies”, the splicing of mouse antibody genes to human antibody genes to obtain a molecule with appropriate antigen specificity and biological activity, can be used. Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Antibodies which specifically bind to SLC22A7 can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to SLC22A7. Antibodies with related specificity, but of distinct idiotypic composition, can be generated by chain shuffling from random combinatorial immunoglobin libraries. Single-chain antibodies also can be constructed using a DNA amplification method, such as PCR, using hybridoma cDNA as a template. Single-chain antibodies can be mono- or bispecific, and can be bivalent or tetravalent. Construction of tetravalent, bispecific single-chain antibodies is taught. A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology.

Antibodies which specifically bind to SLC22A7 also can be produced by inducing in vivo production in the lymphocyte population or by screening immunoglobulin libraries or panels of highly specific binding reagents. Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which SLC22A7 is bound. The bound antibodies can then be eluted from the column using a buffer with a high salt concentration.

### Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of SLC22A7 gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of SLC22A7 gene expression can be obtained by designing antisense oligonucleotides which will form duplexes to the control, 5′, or regulatory regions of the SLC22A7 gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature [Nicholls, (1993)]. An antisense oligonucleotide also can be designed to block translation of mRNA by preventing the transcript from binding to ribosomes.

Precise complementarity is not required for successful complex formation between an antisense oligonucleotide and the complementary sequence of a SLC22A7 polynucleotide. Antisense oligonucleotides which comprise, for example, 2, 3, 4, or 5 or more stretches of contiguous nucleotides which are precisely complementary to a SLC22A7 polynucleotide, each separated by a stretch of contiguous nucleotides which are not complementary to adjacent SLC22A7 nucleotides, can provide sufficient targeting specificity for SLC22A7 mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular SLC22A7 polynucleotide sequence. Antisense oligonucleotides can be modified without affecting their ability to hybridize to a SLC22A7 polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art.

### Ribozymes

Ribozymes are RNA molecules with catalytic activity [Uhlmann, (1987)]. Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a SLC22A7 polynucleotide can be used to generate ribozymes which will specifically bind to mRNA transcribed from a SLC22A7 polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within a SLC22A7 RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate SLC22A7 RNA targets also can be evaluated by testing accessibility to hybridization with complementary oligonucleotides using ribonuclease protection assays. The nucleotide sequences shown in SEQ ID NO: 1 and its complement provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease SLC22A7 expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

For binding assays, the test compound is preferably a small molecule which binds to and occupies the active site of SLC22A7 polypeptide, thereby making the ligand binding site inaccessible to substrate such that normal biological activity is prevented. Examples of such small molecules include, but are not limited to, small peptides or peptide-like molecules.

### Pharmaceutical Compositions

This invention further pertains to novel agents identified by the above-described screening assays and uses thereof for treatments as described herein.

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes pharmaceutical compositions comprising a regulator of SLC22A7 expression or activity (and/or a regulator of the activity or expression of a protein in the SLC22A7 signaling pathway) as well as methods for preparing such compositions by combining one or more such regulators and a pharmaceutically acceptable carrier. Also within the invention are pharmaceutical compositions comprising a regulator identified using the screening assays of the invention packaged with instructions for use. For regulators that are inhibitors of SLC22A7 activity or which reduce SLC22A7 expression, the instructions specify use of the pharmaceutical composition for treatment of disorders of the central nervous system. For regulators that are activators of SLC22A7 activity or increase of SLC22A7 expression, the instructions specify use of the pharmaceutical composition for treatment of cardiovascular diseases, kidney and liver diseases.

An antagonist of SLC22A7 may be produced using methods which are generally known in the art. In particular, purified SLC22A7 may be used to produce antibodies or to screen libraries of pharmaceutical agents to identify those which specifically bind SLC22A7. Antibodies to SLC22A7 may also be generated using methods that are well known in the art. Such antibodies may include, but are not limited to, polyclonal, monoclonal, chimeric, single chain antibodies, Fab fragments, and fragments produced by a Fab expression library. Antibodies like those which bind to an epitope comprising Glutamate at position 441 of SLC22A7 are preferred.

In another embodiment of the invention, the polynucleotides encoding SLC22A7, or any fragment or complement thereof, may be used for therapeutic purposes. In one aspect, the complement of the polynucleotide encoding SLC22A7 may be used in situations in which it would be desirable to block the transcription of the mRNA. In particular, cells may be transformed with sequences complementary to polynucleotides encoding SLC22A7. Thus, complementary molecules or fragments may be used to modulate SLC22A7 activity, or to achieve regulation of gene function. Such technology is now well known in the art, and sense or antisense oligonucleotides or larger fragments can be designed from various locations along the coding or control regions of sequences encoding SLC22A7.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequence complementary to the polynucleotides of the gene encoding SLC22A7. These techniques are described, for example, in [Scott and Smith (1990) Science 249:386-390].

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical composition containing SLC22A7 in conjunction with a pharmaceutically acceptable carrier, for any of the therapeutic effects discussed above. Such pharmaceutical compositions may consist of SLC22A7, antibodies to SLC22A7, and mimetics, agonists, antagonists, or inhibitors of SLC22A7. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### Determination of a Therapeutically Effective Dose

The determination of a therapeutically effective dose is well within the capability of those skilled in the art. A therapeutically effective dose refers to that amount of active ingredient which increases or decreases SLC22A7 activity relative to SLC22A7 activity which occurs in the absence of the therapeutically effective dose. For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 micrograms to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun”, and DEAE- or calcium phosphate-mediated transfection.

If the expression product is mRNA, the reagent is preferably an antisense oligonucleotide or a ribozyme. Polynucleotides which express antisense oligonucleotides or ribozymes can be introduced into cells by a variety of methods, as described above. Preferably, a reagent reduces expression of SLC22A7 gene or the activity of SLC22A7 by at least about 10, preferably about 50, more preferably about 75, 90, or 100% relative to the absence of the reagent. The effectiveness of the mechanism chosen to decrease the level of expression of SLC22A7 gene or the activity of SLC22A7 can be assessed using methods well known in the art, such as hybridization of nucleotide probes to SLC22A7-specific mRNA, quantitative RT-PCR, immunologic detection of SLC22A7, or measurement of SLC22A7 activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects. Any of the therapeutic methods described above can be applied to any subject in need of such therapy, including, for example, mammals such as dogs, cats, cows, horses, rabbits, monkeys, and most preferably, humans.
A method of screening for identifying and/or obtaining a compound capable of modulating SLC22A7 transport activity comprising:
   contacting a test compound with a system for measuring SLC22A7 glutamate transport activity, and
   detecting an altered level of the SLC22A7 transport activity in the presence of the test compound compared to the SLC22A7 transport activity in the absence of the test compound and/or presence of a control,
   wherein the system comprises a SLC22A7 transporter polypeptide or a functional fragment thereof, and one or more of the identified substrates for measuring SLC22A7 transport activity by the system. or
   wherein the system comprises a SLC22A7 transporter polypeptide or a functional fragment thereof, and one or more of the identified substrates comprised in the group consisting of glutamate, orotate and trigonelline and wherein the transport activity is measured using one or more of the aforementioned substrates

A method of screening for therapeutic agents useful in the treatment of a glutamate metabolism disease contacting a test compound with a system for measuring SLC22A7 glutamate transport activity, and
detecting an altered level of the SLC22A7 transport activity in the presence of the test compound compared to the SLC22A7 transport activity in the absence of the test compound and/or presence of a control,
wherein the system comprises a SLC22A7 transporter polypeptide or a functional fragment thereof, and one or more of the identified substrates comprised in the group consisting of glutamate, orotate and trigonelline, or
wherein the system comprises a SLC22A7 transporter polypeptide or a functional fragment thereof, and one or more of the identified substrates comprised in the group consisting of glutamate, orotate and trigonelline and wherein the transport activity is measured using one or more of the aforementioned substrates

A method of screening for therapeutic agents useful in the treatment of a glutamate metabolism disease contacting a test compound with a system for measuring SLC22A7 glutamate transport activity, and
detecting an altered level of the SLC22A7 transport activity in the presence of the test compound compared to the SLC22A7 transport activity in the absence of the test compound and/or presence of a control,
and testing the identified compound in an animal model of the glutamate metabolism disease,
wherein the system comprises a SLC22A7 transporter polypeptide or a functional fragment thereof, and one or more of the identified substrates comprised in the group consisting of glutamate, orotate and trigonelline, or
wherein the system comprises a SLC22A7 transporter polypeptide or a functional fragment thereof, and one or more of the identified substrates comprised in the group consisting of glutamate, orotate and trigonelline and wherein the transport activity is measured using one or more of the aforementioned substrates

Glutamate metabolism diseases are liver, kidney, cardiovascular or CNS diseases.

Preferred substrates are selected from the group consisting of glutamate, L-glutamate, glutamic acid, L-glutamic acid, orotate, orotic acid, trigonelline, their salts and acids, derivatives thereof and isotope labeled compounds. A further preferred substrate is glutamate, even further preferred is L-glutamate. A preferred salt is a soluble salt e.g. a sodium salt.

The method of the foregoing embodiments, wherein the compound enhances the transporter function of SLC22A7.

The method of the foregoing embodiments, wherein the compound reduces or blocks the transporter function of SLC22A7.

A method of screening for identifying and/or obtaining a compound for the treatment and/or prophylaxis of a disease related to glutamate metabolism
contacting a test compound with a system for measuring SLC22A7 glutamate transport activity, which system comprises an SLC22A7 polypeptide or a functional fragment thereof, and a substrate for measuring SLC22A7 transport activity by the system; and
detecting an altered level of the SLC22A7 glutamate transport activity of the SLC22A7 polypeptide or functional fragment in the presence of the test compound compared to the SLC22A7 transport activity in the absence of the test compound and/or presence of a control.

The method of any one of the foregoing embodiments, wherein the compound is a small molecule.

The method of any one of the foregoing embodiments, wherein the compound binds intracellular or extracellular to the SLC22A7 polypeptide.

The method of any one of the foregoing embodiments, wherein the substrate for measuring SLC22A7 transport is selected from the group consisting of glutamate or a derivative or analog of any one thereof.

The method of any one of the foregoing embodiments, wherein said system comprises a liposome or cell based assay.

The method of the foregoing embodiments, wherein the cells of said cell based assay express a recombinant SLC22A7 polypeptide or a functional fragment of an SLC22A7 polypeptide.

The method of the foregoing embodiments, wherein said cells are genetically engineered to (over) express or inhibit the expression of the SLC22A7 gene.

The method of any one of the foregoing embodiments, wherein said cells are cells of the cardiovascular, liver, kidney or CNS system.

A method of screening for identifying and/or obtaining a compound for treating a disease related to the glutamate level, which method comprises:
providing a transgenic animal or a mutant animal, which animal expresses a variant SLC22A7 gene, due to the deregulation of the glutamate level in cells or tissue of said animal compared to cells or tissue of a corresponding wild type or control animal;
contacting the animal with a test compound; and
detecting an improvement in a condition of the animal in response to the test compound, wherein the condition is a symptom of a disorder of the liver, kidney, cardiovascular system or CNS.

The method of any one of the foregoing embodiments, wherein said contacting step further includes contacting said system or animal with at least one second test substance in the presence of said first test substance.

The method of any one of the foregoing embodiments, wherein a compound known to activate or inhibit SLC22A7 activity is added to the system or administered to the animal.

The method of any one of the foregoing embodiments, wherein the test substance is a therapeutic agent.

The method of any one of the foregoing embodiments, wherein the test substance is a mixture of therapeutic agents.

The method of any one of the foregoing embodiments, wherein preferably in a first screen said test substance is comprised in and subjected as a collection of test substances.

The method of any one of the foregoing embodiments, which is performed on an array.

The method of any one of the foregoing embodiments, wherein said system is contained in a container.

The method of any one of the foregoing embodiments, wherein said container is a well in a microtiter plate.

A method of obtaining and manufacturing a drug comprising the steps of the method of any one of the foregoing embodiments.

The method of the foregoing embodiments, wherein an enhanced or reduced level or activity of the SLC22A7 transporter is indicative for the drug.

A method of determining toxicity of a compound comprising the steps of the method of any one of the foregoing embodiments, wherein a reduced or enhanced level or activity of the SLC22A7 is indicative for the efficacy of the compound.

The method of any one of the foregoing embodiments further comprising modifying said substance to alter, eliminate and/or derivatize a portion thereof suspected causing toxicity, increasing bioavailability, solubility and/or half-life.

The method of any one of the foregoing embodiments further comprising mixing the substance isolated or modified with a pharmaceutically acceptable carrier.

Use of glutamate or a derivative or analog thereof, an SLC22A7 polypeptide or functional fragment thereof, a nucleic acid molecule encoding said SLC22A7 polypeptide or functional fragment thereof for use in a method of any one of the foregoing embodiments.

Use of a compound which inhibits glutamate transport activity of an SLC22A7 polypeptide for the manufacture of a medicament for the treatment and/or prophylaxis of a disease related to the CNS system.

Use of a compound which activates glutamate transport activity of an SLC22A7 polypeptide for the manufacture of a medicament for the treatment and/or prophylaxis of a disease related to the kidney, liver, or cardiovascular system.

The use of the foregoing embodiments, wherein the disease is a liver disease selected from the group consisting of:

Liver diseases comprise primary or secondary, acute or chronic diseases or injury of the liver which may be acquired or inherited, benign or malignant, and which may affect the liver or the body as a whole. They comprise but are not limited to disorders of the bilirubin metabolism, jaundice, syndroms of Gilbert's, Crigler-Najjar, Dubin-Johnson and Rotor; intrahepatic cholestasis, hepatomegaly, portal hypertension, ascites, Budd-Chiari syndrome, portal-systemic encephalopathy, fatty liver, steatosis, Reye's syndrome, liver diseases due to alcohol, alcoholic hepatitis or cirrhosis, fibrosis and cirrhosis, fibrosis and cirrhosis of the liver due to inborn errors of metabolism or exogenous substances, storage diseases, syndromes of Gaucher's, Zellweger's, Wilson's—disease, acute or chronic hepatitis, viral hepatitis and its variants, inflammatory conditions of the liver due to viruses, bacteria, fungi, protozoa, helminths; drug induced disorders of the liver, chronic liver diseases like primary sclerosing cholangitis, alpha1-antitrypsin-deficiency, primary biliary cirrhosis, postoperative liver disorders like postoperative intrahepatic cholestasis, hepatic granulomas, vascular liver disorders associated with systemic disease, benign or malignant neoplasms of the liver, disturbance of liver metabolism in the new-born or prematurely born.

The use of the foregoing embodiments, wherein said cardiovascular disease is selected from the group consisting of:

Heart failure is defined as a pathophysiological state in which an abnormality of cardiac function is responsible for the failure of the heart to pump blood at a rate commensurate with the requirement of the metabolizing tissue. It includes all forms of pumping failures such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina and asymptomatic ischemia.

Arrhythmias include all forms of atrial and ventricular tachyarrhythmias, atrial tachycardia, atrial flutter, atrial fibrillation, atrio-ventricular reentrant tachycardia, preexitation syndrome, ventricular tachycardia, ventricular flutter, ventricular fibrillation, as well as bradycardic forms of arrhythmias.

Hypertensive vascular diseases include primary as well as all kinds of secondary arterial hypertension, renal, endocrine, neurogenic, others. The genes may be used as drug targets for the treatment of hypertension as well as for the prevention of all complications arising from cardiovascular diseases.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon and venous disorders.

Atherosclerosis is a cardiovascular disease in which the vessel wall is remodeled, compromising the lumen of the vessel. The atherosclerotic remodeling process involves accumulation of cells, both smooth muscle cells and monocyte/macrophage inflammatory cells, in the intima of the vessel wall. These cells take up lipid, likely from the circulation, to form a mature atherosclerotic lesion. Although the formation of these lesions is a chronic process, occurring over decades of an adult human life, the majority of the morbidity associated with atherosclerosis occurs when a lesion ruptures, releasing thrombogenic debris that rapidly occludes the artery. When such an acute event occurs in the coronary artery, myocardial infarction can ensue, and in the worst case, can result in death.

The formation of the atherosclerotic lesion can be considered to occur in five overlapping stages such as migration, lipid accumulation, recruitment of inflammatory cells, proliferation of vascular smooth muscle cells, and extracellular matrix deposition. Each of these processes can be shown to occur in man and in animal models of atherosclerosis, but the relative contribution of each to the pathology and clinical significance of the lesion is unclear.

Thus, a need exists for therapeutic methods and agents to treat cardiovascular pathologies, such as atherosclerosis and other conditions related to coronary artery disease.

Cardiovascular diseases include but are not limited to disorders of the heart and the vascular system like congestive heart failure, myocardial infarction, ischemic diseases of the heart, all kinds of atrial and ventricular arrhythmias, hypertensive vascular diseases, peripheral vascular diseases, and atherosclerosis.

Too high or too low levels of fats in the bloodstream, especially cholesterol, can cause long-term problems. The risk to develop atherosclerosis and coronary artery or carotid artery disease (and thus the risk of having a heart attack or stroke) increases with the total cholesterol level increasing. Nevertheless, extremely low cholesterol levels may not be healthy. Examples of disorders of lipid metabolism are hyperlipidemia (abnormally high levels of fats (cholesterol, triglycerides, or both) in the blood, may be caused by family history of hyperlipidemia), obesity, a high-fat diet, lack of exercise, moderate to high alcohol consumption, cigarette smoking, poorly controlled diabetes, and an underactive thyroid gland), hereditary hyperlipidemias (type I hyperlipoproteinemia (familial hyperchylomicronemia), type II hyperlipoproteinemia (familial hypercholesterolemia), type III hyperlipoproteinemia, type IV hyperlipoproteinemia, or type V hyperlipoproteinemia), hypolipoproteinemia, lipidoses (caused by abnormalities in the enzymes that metabolize fats), Gaucher's disease, Niemann-Pick disease, Fabry's disease, Wolman's disease, cerebrotendinous xanthomatosis, sitosterolemia, Refsum's disease, or Tay-Sachs disease.

Kidney disorders may lead to hypertension or hypotension. Examples for kidney problems possibly leading to hypertension are renal artery stenosis, pyelonephritis, glomerulonephritis, kidney tumors, polycistic kidney disease, injury to the kidney, or radiation therapy affecting the kidney. Excessive urination may lead to hypotension.

The use of the foregoing embodiment, wherein said CNS disease is selected from the group consisting of CNS disorders include disorders of the central nervous system as well as disorders of the peripheral nervous system.

CNS disorders include, but are not limited to brain injuries, cerebrovascular diseases and their consequences, Parkinson's disease, corticobasal degeneration, motor neuron disease, dementia, including ALS, multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld-Jakob dementia, HIV dementia, schizophrenia with dementia, and Korsakoff's psychosis, within the meaning of the definition are also considered to be CNS disorders.

Similarly, cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities are also considered to be CNS disorders.

Pain, within the meaning of this definition, is also considered to be a CNS disorder. Pain can be associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation). Non-central neuropathic pain includes that associated with post mastectomy pain, phantom feeling, reflex sympathetic dystrophy (RSD), trigeminal neuralgiara-dioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia. Pain associated with peripheral nerve damage, central pain (i.e. due to cerebral ischemia) and various chronic pain i.e., lumbago, back pain (low back pain), inflammatory and/or rheumatic pain. Headache pain (for example, migraine with aura, migraine without aura, and other migraine disorders), episodic and chronic tension-type headache, tension-type like headache, cluster headache, and chronic paroxysmal hemicrania are also CNS disorders.

Visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome and protatodynia are also CNS disorders.

Also considered to be a disorder of the nervous system are acute pain, for example postoperative pain, and pain after trauma.

The use of the foregoing embodiments, wherein said kidney disease is selected from the group consisting of kidney disorders including acute and chronic kidney diseases as (but not limited to): acute kidney failure, acute nephritic syndrome, analgesic nephropathy, atheroembolic renal disease, chronic kidney failure, chronic nephritis, congenital nephrotic syndrome, end-stage renal disease, goodpasture syndrome, interstitial nephritis, kidney cancer, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephropathy—IgA, nephrosis (nephrotic syndrome), polycystic kidney disease, post-streptococcal GN, reflux nephropathy, renal artery embolism, renal artery stenosis, renal disorders, renal papillary necrosis, renal tubular acidosis type I, renal tubular acidosis type II, renal underperfusion, renal vein thrombosis

The use of any one of the foregoing embodiments, wherein the pharmaceutical composition is designed to be administered by oral administration or by intravitreal, intramuscular, intravenous, intraperitoneal, intrathecal, intraventricular or intracranial injection.

### EXAMPLES

### Example 1

### Generation of SLC22A7 Cells

### Plasmid Constructs

The cDNAs of OAT2 from human (OAT2h) and rat (OAT2r), OAT1 from human (OAT1h), and the cDNA coded by the human SLC22AI 3 gene were generated by RT-PCR, cloned into pUC19, fully sequenced, and inserted into expression vector pEBTetD. pEBTetD is an episomal Epstein-Barr plasmid vector for doxycycline-inducible protein expression in human cell lines based on the simple tetracycline repressor (Bach M et al. FEBS Journal 2007; 274:783-790.). With e.g. the gluatmine transporter (SLC22A7), this system provides a high rate of carrier-mediated transport in the on-state (doxy(+)) (=100%) and a low rate (4%) in the off-state (doxy(−)) (=leak expression) (Bach M et al. FEBS Journal 2007; 274:783-790.). The amino acid sequence of OAT2h corresponds to GenBank entry NM_{—}006672. The 5′-interface between pEBTetD and cDNA is given under SEQ ID NO:3; the 3′-interface is given in SEQ ID NO4. The amino acid sequence of OAT2r corresponds to GenBank entry NM_{—}053537. The 5′-interface is given in SEQ ID NO5. The 3′-UTR corresponds to GenBank entry L27651; the 3′-interface is given in SEQ ID NO 6. The amino acid sequence of OAT1h corresponds to GenBank entry NM_{—}153276. The 5′-interface is given in SEQ ID NO 7; the 3′-interface is given in SEQ ID NO 8. The amino acid sequence of SLC22A13h corresponds to GenBank entry NM_{—}004256. The 5′-interface is given in SEQ ID NO 9; the 3′-interface is given in SEQ ID NO 10. The OAT2h mutant was generated with the QuikChange® II Kit (Stratagene, Agilent Technologies, Waldbronn, Germany).

### Generation of Inducible SLC22A7 Cell Line

The cDNA of SLC22A7 was inserted into the eukaryotic expression vector pcDNA5/FRT/TO (Invitrogen) to yield pcDNA5/FRT/TO/SLC22A7h. This plasmid was cotransfected together with plasmid pOG44 by lipofection with Tfx-50 (Promega) into Flp-In T-REx 293 cells (Invitrogen; referred to as 293-FIT-NT in the remainder). After antibiotic selection with hygromycin B and blasticidin S, the surviving cells, used as a pool and designated as 293-FIT-SLC22A7h, were assayed for SLC22A7 transcripts by Northern analysis. In the presence of 1 μg/ml doxycycline in the growth medium for 20-48 h to turn on transcription, the SLC22A7 mRNA was about 100-fold more abundant than in the off-state without doxycycline in the medium.

### Cell Culture

293 cells (ATCC CRL-1573; also known as HEK-293 cells), a transformed cell line derived from human embryonic kidney, were grown at 37° C. in a humidified atmosphere (5% CO2) in plastic culture flasks (Falcon 3112, Becton Dickinson, Heidelberg, Germany). The growth medium was Dulbecco's Modified Eagle Medium (Life Technologies 31885-023, Invitrogen, Karlsruhe, Germany) supplemented with 10% fetal calf serum (PAA Laboratories, Cölbe, Germany). Medium was changed every 2-3 days and the culture was split every 5 days. Stably transfected cell lines were generated as reported previously (Bach M et al. FEBS Journal 2007; 274:783-790.). As there is no integration of vector pEBTetD into the genome, clonal isolation of transfected cells is not necessary; we thus use cell pools rather than single cell clones. Cell culture medium always contained 3 μg/ml puromycin (PAA Laboratories) to ascertain plasmid maintenance. To turn on protein expression, cells were cultivated for at least 20 h in regular growth medium supplemented with 1 μg/ml doxycycline (195044, MP Biomedicals, Eschwege, Germany).

### Expression of SLC22A7 and Overexpressing Cell Lines

Expression of SLC22A7 is accomplished by subcloning the cDNAs into appropriate expression vectors and transfecting the vectors into expression hosts such as, e.g., E. coli. In a particular case, the vector is engineered such that it contains a promoter for β-galactosidase, upstream of the cloning site, followed by sequence containing the amino-terminal Methionine and the subsequent seven residues of β-galactosidase. Immediately following these eight residues is an engineered bacteriophage promoter useful for artificial priming and tran [symbol] scription and for providing a number of unique endonuclease restriction sites for cloning.

Induction of the isolated, transfected bacterial strain IPTG using standard methods produces a fusion protein corresponding to the first seven residues of β-galactosidase, about 15 residues of “linker”, and the peptide encoded within the cDNA. Since cDNA clone inserts are generated by an essentially random process, there is probability of 33% that the included cDNA will lie in the correct reading frame for proper translation. If the cDNA is not in the proper reading frame, it is obtained by deletion or insertion of the appropriate number of bases using well known methods including in vitro mutagenesis, digestion with exonuclease III or mung bean nuclease, or the inclusion of an oligonucleotide linker of appropriate length.

The SLC22A7 cDNA is shuttled into other vectors known to be useful for expression of proteins in specific hosts. Oligonucleotide primers containing cloning sites as well as a segment of DNA (about 25 bases) sufficient to hybridize to stretches at both ends of the target cDNA is synthesized chemically by standard methods. These primers are then used to amplify the desired gene segment by PCR. The resulting gene segment is digested with appropriate restriction enzymes under standard conditions and isolated by gel electrophoresis. Alternately, similar gene segments are produced by digestion of the cDNA with appropriate restriction enzymes. Using appropriate primers, segments of coding sequence from more than one gene are ligated together and cloned in appropriate vectors. It is possible to optimize expression by construction of such chimeric sequences.

Suitable expression hosts for such chimeric molecules include, but are not limited to, mammalian cells such as Chinese Hamster Ovary (CHO) and human 293 cells, insect cells such as Sf9 cells, yeast cells such as Saccharomyces cerevisiae and bacterial cells such as E. coli. For each of these cell systems, a useful expression vector also includes an origin of replication to allow propagation in bacteria, and a selectable marker such as the β-lactamase antibiotic resistance gene to allow plasmid selection in bacteria. In addition, the vector may include a second selectable marker such as the neomycin phosphotransferase gene to allow selection in transfected eukaryotic host cells. Vectors for use in eukaryotic expression hosts require RNA processing elements such as 3′ polyadenylation sequences if such are not part of the cDNA of interest.

Additionally, the vector contains promoters or enhancers which increase gene expression. Such promoters are host specific and include MMTV, SV40, and metallothionine promoters for CHO cells; trp, lac, tac and T7 promoters for bacterial hosts; and alpha factor, alcohol oxidase and PGH promoters for yeast. Transcription enhancers, such as the rous sarcoma virus enhancer, are used in mammalian host cells. Once homogeneous cultures of re-combinant cells are obtained through standard culture methods, large quantities of re-combinantly produced SLC22A7 are recovered from the conditioned medium and analyzed using chromatographic methods known in the art. For example, SLC22A7 can be cloned into the expression vector pcDNA3, as exemplified herein. This product can be used to transform, for example, HEK293 or COS by methodology standard in the art. Specifically, for example, using Lipofectamine (Gibco BRL catolog no. 18324-020) mediated gene transfer.

### Example 2

### Transport Assays

For measurement of solute uptake, cells were grown in surface culture on 60 mm polystyrol dishes (Nunclon 150288, Nunc, Roskilde, Denmark) precoated with 0.1 g/l poly-L-ornithine in 0.15 M boric acid-NaOH, pH 8.4. Cells were used for uptake experiments at a confluence of at least 70%. Uptake was measured at 37° C. Uptake buffer contains 125 mmol/l NaCl, 25 mmol/l HEPES-NaOH pH 7.4, 5.6 mmol/l (+)glucose, 4.8 mmol/l KCl, 1.2 mmol/l KH2PO4, 1.2 mmol/l CaCl2, and 1.2 mmol/l MgSO4. In glutamate efflux experiments, uptake buffer without KH2PO4 was used to avoid MS interference. After preincubation for at least 20 minutes in 4 ml of uptake buffer, the buffer was replaced with 2 ml of substrate in uptake buffer. The total substrate concentration if not indicated otherwise was 0.1 μmol/l for radiotracer assays and 10 μmol/l for unlabeled compounds. Incubation was stopped after 1 min by rinsing the cells four times each with 4 ml ice-cold uptake buffer. Radioactivity was determined, after cell lysis with 0.1% v/v Triton X-100 in 5 mmol/l TRIS-HCl pH 7.4, by liquid scintillation counting. For LCelectrospray ionization-MS/MS analysis, cells were lysed with methanol and stored at −20° C.

After centrifugation (1 min, 16000×g, 20° C.) of thawed lysates, 100 μl of the supernatant was mixed with either 10 μl unlabeled MPP+ iodide (0.5 ng/μl) or biphenyl-4-carboxylic acid (Sigma-Aldrich) as internal standard. Of this mixture, 201 samples were analyzed by LCMS/MS on a triple quadrupole mass spectrometer (4000 Q TRAP, Applied Biosystems, Darmstadt, Germany). The following LC conditions were used: cGMP, Atlantis dC18 column (particle size 5 Gm, diameter×length=3.0×100 mm; Waters, Eschborn, Germany); A: 10 mM formic acid, B: 10 mM formic acid in acetonitrile; gradient: 0.3 ml/min, 5% B at 0 min, 5% B at 2 min, 60% B at 5 min, 5% B at 7 min, stop at 10 min; glutamate, Atlantis dC18 column; A: 0.1% formic acid, B: 0.1% formic acid in acetonitrile; gradient: 0.3 ml/min, 20% B at 0 min, 80% B at 4 min, 80% B at 5 min, 20% B at 8 min, stop at 8.5 min; orotic acid, XBridge Shield RP18 column (3.5 m, 3.0×100 mm; Waters); A: 10 mM ammonium acetate pH 8.9, B: methanol; gradient: 0.3 ml/min, 10% B at 0 min, 80% B at 5.5 min, 10% B at 8 min, stop at 9 min; PAH, Atlantis HILIC Silica column (5 μm, 3.0×50 mm; Waters); 10 mM ammonium acetate pH 4.5, B: methanol; gradient: 0.3 ml/min, 90% B at 0 min, 10% B at 4 min, 10% B at 5 min, 90% B at 8 min, stop at 9 min; trigonelline, Atlantis HILIC Silica column; A: 0.1% formic acid, B: 0.1% formic acid in methanol; gradient: 0.4 ml/min; 90% B at 0 min, 10% B at 4 min, 10% B at 5 min, 90% B at 8 min, stop at 10 min. Atmospheric pressure ionization with positive or negative electrospray was used. For quantification (scan time 150 ms), the optimal collision energy for argon-induced fragmentation in the second quadrupole was determined for each analyte. From the product ion spectra, the following fragmentations were selected for selected reaction monitoring (m/z parent, m/z fragment, collision energy (V), ion detection: N/P=negative/positive): cGMP: 346, 152, 29, P; glutamate: 146, 102, −20, N; MPP+: 170, 128, 43, P; orotic acid: 155, 111, −16, N; PAH: 195, 120, 15, P; trigonelline: 138, 79, 41, P. For each analyte, the area of the intensity vs. time peak was integrated and divided by the area of the internal standard peak to yield the analyte response ratio. Linear calibration curves were constructed from at least six standards which were prepared using control cell lysates as solvent. Sample analyte content was calculated from the analyte response ratio and the slope of the calibration curve, obtained by linear regression. With LC-MS quantitation, solute content of cell lysates was determined for 4 conditions (paired dishes, incubation time 1 min): a) transporter expression on, uptake buffer; b) expression off, uptake buffer; c) expression on, substrate in uptake buffer; d) expression off, substrate in uptake buffer. Acute uptake mediated by heterologously expressed carrier was then calculated as (c−a)−(d−b). This approach takes into account endogenous solute content and non-specific uptake. Protein was measured by the BCA assay (Pierce) with bovine serum albumin as standard. The protein content of MS samples was estimated from 3 matched cell dishes.

### LC-MS Difference Shading

In this method, lysates of cells with or without transporter expression are analyzed by fullscan LC-MS. From these data sets, gray scale images with axes of m/z and time are generated in which low intensities are rendered black and high intensities are rendered white. Finally, a difference image is created based on RGB pixel information, combining the red channel from the transporter active image with the green and blue channels from the transporter inactive image. Thus, compounds only present in the active or inactive data set can be spotted as red or cyan signals, respectively, while compounds present in equal amounts in both sets remain scales of gray.

### Example 3

### Expression Profiling by Real-Time PCR

For relative quantitation of OAT2h mRNA levels in human cells and tissues, a TaqMan™ realtime PCR assay was employed on a 7900 HT Sequence Detection system (Applied Biosystems, Darmstadt, Germany) according to the manufacturer's protocols. For first strand cDNA synthesis, 85 μg of total RNA was incubated for 1.5 h at 37° C. with 2 U/μl Omniscript reverse transcriptase (Qiagen, Hilden, Germany) in the supplied buffer plus 9.5 μM random hexamer primer, 0.5 mM per dNTP, and 3000 U RNaseOUT™ (Invitrogen) in a final volume of 680 μl. The resulting cDNA was diluted 1:10 with water and directly used as template in PCR. A PCR reaction (20 μl) contained, in addition to 5 μl cDNA and 10 μl qPCR MasterMix Plus (Eurogentec, Seraing, Belgium), 0.2 M per OAT2h amplification primer (forward: SEQ ID NO 11; reverse: SEQ ID NO 12), and 0.2 μM FAM™/TAMRA™-labeled OAT2h probe (SEQ ID NO 13; designed to cross exon boundaries). The thermal protocol was set to 2 min at 50° C., followed by 10 min at 95° C., followed by 40 cycles of 15 s at 95° C. and 1 min at 60° C. To normalize the amount of cDNA per assay, the expression of multiple housekeeping genes (e.g. hypoxanthine phosphoribosyltransferase, glyceraldehyde-3-phosphate dehydrogenase, and β-actin) was measured in parallel assays. Relative expression of OAT2h was then calculated using the normalized expression values.

### Laser Capture Microdissection

Rat liver sections (16 μm) were prepared using a cryostat at −20° C. and mounted on RNase-free PEN-membrane slides (Leica Microsystems, Germany). Sections were dried briefly at room temperature, fixed in 75% and 100% ethanol (cooled to −20° C.) for 15 s each, and stained with Weigert's iron hematoxylin followed by alcoholic eosin Y solution (Sigma-Aldrich, Germany). A Leica DM6000B LMD system (Leica Microsystems) was used to isolate circular periportal and pericentral areas (average area=0.02 mm2). RNA was isolated from the captured discs using the RNeasy Plus Micro Kit (Qiagen, Germany). RNA integrity was verified using the Agilent 2100 bioanalyzer and RNA 6000 Pico LabChip Kit (Agilent Technologies, Germany). cDNA was generated with the High Capacity cDNA Reverse Transcription Kit (Applied Biosystems, Germany) and analyzed by real-time PCR on a LightCycler 1.0 apparatus with system 2.0 software (Roche, Mannheim, Germany). Product accumulation was detected with locked nucleic acid hydrolysis probes (TaqMan principle) from the human Universal ProbeLibrary (Roche). A single reaction (total volume 101) contained 11 master mix (5× concentration; LightCycler TaqMan Master; Roche 04735536001), 1 μmol/l each of forward and reverse primer, 50 nmol/l probe, and 11 μl of cDNA. Contamination controls contained water instead of DNA. After enzyme activation (10 min, 95° C.), thermocycling consisted of 45 cycles of 10 s at 95° C., 30 s at 55° C., and 1 s at 72° C.; velocity of temperature change was 20° C./s.

Fluorescence curves were analyzed by non-linear simultaneous fitting as described previously to yield the relative mRNA level; β-actin was used for normalization.

### Example 3

### Transporter Assay

In a transporter assay a sample which can be a chemical compound or an antibody acting as channel blocker or transport inhibitor, is reacted in a reaction mixture simultaneously or in succession with an adhesive cell culture expressing the transporter of interest. A part of the experiment is also a compound or peptide labeled radiochemically either with a tritium or 125-iodine label known to be specifically transported by the transporter of interest through the cell membrane.

First, a cell line expressing the transporter is cultured in an appropriate container (eg. 96 well plate for scintillation counting) and with an appropriate growth medium at an optimal cell density and temperature. Then, to determine the transport blocking or inhibiting properties of compounds, the growth medium is replaced by a buffer, eg. PBS containing the compounds or antibodies at a fixed or varying concentration. After an specific time the buffer is replaced by a buffer containing the radiolabeled compound and incubated again for a specific time. Only if the transporter has not been blocked by the compounds, the radiolabeled compound is transported into the cells.

To determine the amount of radiolabeled compound transported into the cell, the buffer is removed and the cells are washed several times with a buffer without the radiolabeled compound. Finally the buffer is removed and replaced by cell lysis buffer and a scintillation fluid. The container is then counted in an appropriate scintillation counter.

### Example 4

### Binding Assay

In a receptor binding assay a sample which can be a chemical compound acting as an agonist or antagonist or an antibody acting as an antagonist, is reacted in a reaction mixture simultaneously or in succession with a receptor membrane preparation. A part of the reaction mix is also a compound or peptide labelled radiochemically either with a tritium or 125-iodine label known to bind specifically to the transporter.

First, the receptor membrane preparation is mixed in an appropriate buffer with compounds or antibodies at varying concentrations for which the IC50 value is going to be determined. The transporter/compound or antibody complex is incubated for a specific time until a steady state of binding and dissociation has formed. Then, the radiolabeled compound or peptide is added to the reaction mix. The radiolabeled compound and the non-radiolabelled compounds/antibodies compete for the binding site of the transporter.

After reaching the steady state, the unbound radiolabeled compound/peptide is separated from the receptor bound radiolabeled compound/peptide by means of filtration and subsequent washing with an appropriate buffer. The transporter membrane/radiolabeled compound complex is bound to the filtration membrane, which is dried and an appropriate scintillator is added so the radioactive signal can be recorded by a suitable counter.

Alternatively the bound and unbound separation is achieved by binding of the transporter membrane/compound complex to specific beads in a scintillation proximity assay (SPA). Only by binding of the receptor bound radiolabeled compound in a close proximity to the scintillation beads a scintillation signal can be recorded by a suitable counter. Radiolabeled compounds not in such a close proximity as the transporter membrane/compound complex don't give a signal.

### REFERENCES

1. Mizuno et al., Pharmacol. Rev. 55 (2003), 425-461.
2. Bach M et al. FEBS Journal 2007; 274:783-790.
3. Parrilla et al., Pflugers Arch 1976; 362:49-54.
4. Brosnan et al. Biochem J 1970; 117:91-96.
5. Geerts et al. J Histochem Cytochem 1997; 45:1217-1229.
6. Bacher et al. Biochim Biophys Acta 2009; 1788:2594-2602.
7. Newsholme et al. Cell Biochem Funct 2003; 21:1-9.
8. Clemmesen et al. Gastroenterology 2000; 118:1131-1139.
9. Strauss et al. Gastroenterology 2001; 121:1109-1119.
10. Schmidt et al. Scand J Gastroenterol 2004; 39:974-980.
11. Poeze et al. Clin Nutr 2008; 27:523-530.
12. Meijer et al. Physiol Rev 1990; 70:701-748.
13. Watford M. J Nutr 2000; 130:983 S-987S.
14. Olde et al. Metab Brain Dis 2009; 24:169-181.
15. Souba et al. JPEN J Parenter Enteral Nutr 1990; 14:68 S-70S.
16. Hoogland et al. Neurochem Int 2005; 47:499-506.

1. A method of screening for identifying and/or obtaining a compound capable of modulating SLC22A7 transport activity comprising:
a. contacting a test compound with a system for measuring SLC22A7 transport activity, and
b. detecting an altered level of the SLC22A7 transport activity in the presence of the test compound compared to the SLC22A7 transport activity in the absence of the test compound,
wherein the system comprises a SLC22A7 transporter polypeptide or a functional fragment thereof, and one or more of the identified substrates comprised in the group consisting of glutamate, orotate and trigonelline and wherein the transport activity is measured using one or more of the aforementioned substrates

2. A method according to claim 1 further comprising a step of testing the compound identified in step b in an animal CNS, liver, cardiovascular or kidney disease model for alleviation of disease symptoms.

3. A method according to claim 1, wherein the substrates are isotope labeled.

4. A method according to claim 1, wherein the system comprises a liposom containing or a cell expressing SLC22A7 or a functional fragment thereof.

5. A method according to claim 4, wherein the cell expresses recombinant SLC22A7.

6. A method according to claim 4, wherein the cell endogenously expresses SLC22A7.

7. A method according to claim 4, wherein the cell is of liver, kidney, cardiovascular or CNS origin.

8. (canceled)

9. An antibody or antigen binding fragment thereof specifically binding to a SLC22A7 polypeptide epitope comprising glutamate at position 441, or a ribozyme or an antisense DNA specifically binding to SLC22A7 polynucleotide for the treatment of a CNS disease.

10. A transgenic animal comprising a SLC22A7 variant with increased or decreased transport capacity.

11. An animal according to claim 10, wherein the variant with decreased transport capacity is a variant comprising any another aminoacid than glutamate at position 441.

12. An animal according to claim 10, wherein the variant with increased transport capacity is a variant comprising an insertion of serine and glutamine between position 131 and 132 (E131_W132insSQ).

13. Use of an animal according to claim 10 in an liver, kidney, cardiovascular or CNS disease model.

14. A compound identified by a method according to claim 1.


SEQUENCE LISTING (13 sequences)

SEQ ID NO: 1 - DNA, 2136 bp, Homo sapiens
ccacctccagagtccaagggtctatgtggtgggcagtttgagctggctggatactagagggaggctgcacctgaagcatttggtgggtgagcagcatgggctttgaggagctgctggagcaggtgggcggctttgggcccttccaactgcggaatgtggcactgctggccctgccccgagtgctgctaccactgcacttcctcctgcccatcttcctggctgccgtgcctgcccaccgatgtgccctgccgggtgcccctgccaacttcagccatcaggatgtgtggctggaggcccatcttccccgggagcctgatggcacgctcagctcctgcctccgctttgcctatccccaggctctccccaacaccacgttgggggaagaaaggcagagccgtggggagctggaggatgaacctgccacagtgccctgctctcagggctgggagtacgaccactcagaattctcctctaccattgcaactgagtcccagtgggatctggtgtgtgagcagaaaggtctgaacagagctgcgtccactttcttcttcgccggtgtgctggtgggggctgtggcctttggatatctgtccgacaggtttgggcggcggcgtctgctgctggtagcctacgtgagtaccctggtgctgggcctggcatctgcagcctccgtcagctatgtaatgtttgccatcacccgcacccttactggctcagccctggctggttttaccatcatcgtgatgccactggagctggagtggctggatgtggagcaccgcaccgtggctggagtcctgagcagcaccttctggacagggggcgtgatgctgctggcactggttgggtacctgatacgggactggcgatggcttctgctagctgtcaccctgccttgtgccccaggcatcctcagcctctggtgggtgcctgagtctgcacgctggcttctgacccaaggccatgtgaaagaggcccacaggtacttgctccactgtgccaggctcaatgggcggccagtgtgtgaggacagcttcagccaggaggctgtgagcaaagtggccgccggggaacgggtggtccgaagaccttcatacctagacctgttccgcacaccacggctccgacacatctcactgtgctgcgtggtggtgtggttcggagtgaacttctcctattacggcctgagtctggatgtgtcggggctggggctgaacgtgtaccagacacagctgttgttcggggctgtggaactgccctccaagctgctggtctacttgtcggtgcgctacgcaggacgccgcctcacgcaagccgggacactgctgggcacggccctggcgttcggcactagactgctagtgtcctccgatatgaagtcctggagcactgtcctggcagtgatggggaaagctttttctgaagctgccttcaccactgcttacctgttcacttcagagttgtaccctacggtgctcagacagacagggatggggctgactgcactggtgggccggctggggggctctttggccccactggcggccttgctagatggagtgtggctgtcactgcccaagcttacttatggggggatcgccctgctggctgccggcaccgccctcctgctgccagagacgaggcaggcacagctgccagagaccatccaggacgtggagagaaagagtgccccaaccagtcttcaggaggaagagatgcccatgaagcaggtccagaactaagtgggagtggaggcaggccctccacagaagctctgcagcaggggctgggagagcagaagggcaggcccttcaactcaggctgggagagcagaagggcaggccctgcaactcaggctgggagtatcgaaccctctgcctagggccggagttgctgccagtacccgctccctctgctcatccatccttgattatttggcttctaggaacagttgacttcccagaatgcagtgggctgctgggcacccctctcacggttggggaggattctgtaaataaaggtgccccttgggttggggcagtggtgacgagctgtgggaagagccctggataggaagccactgagtctgccctgggctctgataaaacttcaccattaaaaaaaaaaaaaaaaaaa

SEQ ID NO: 2 - protein, 548 aa, Homo sapiens
MGFEELLEQVGGFGPFQLRNVALLALPRVLLPLHFLLPIFLAAVPAHRCALPGAPANFSHQDVWLEAHLPREPDGTLSSCLRFAYPQALPNTTLGEERQSRGELEDEPATVPCSQGWEYDHSEFSSTIATESQWDLVCEQKGLNRAASTFFFAGVLVGAVAFGYLSDRFGRRRLLLVAYVSTLVLGLASAASVSYVMFAITRTLTGSALAGFTIIVMPLELEWLDVEHRTVAGVLSSTFWTGGVMLLALVGYLIRDWRWLLLAVTLPCAPGILSLWWVPESARWLLTQGHVKEAHRYLLHCARLNGRPVCEDSFSQEAVSKVAAGERVVRRPSYLDLFRTPRLRHISLCCVVVWFGVNFSYYGLSLDVSGLGLNVYQTQLLFGAVELPSKLLVYLSVRYAGRRLTQAGTLLGTALAFGTRLLVSSDMKSWSTVLAVMGKAFSEAAFTTAYLFTSELYPTVLRQTGMGLTALVGRLGGSLAPLAALLDGVWLSLPKLTYGGIALLAAGTALLLPETRQAQLPETIQDVERKSAPTSLQEEEMPMKQVQN

SEQ ID NO: 3 - protein, 37 aa, Artificial Sequence
  primer
GTTTAAACTTAAGCTTGCCACCATGGGCTTTGAGGAG

SEQ ID NO: 4 - DNA, 25 bp, Artificial Sequence
  primer
gtccagaactaactcgagcgatcgc

SEQ ID NO: 5 - DNA, 37 bp, Artificial Sequence
  primer
gtttaaacttaagcttgccaccatgggcttcgaagac

SEQ ID NO: 6 - DNA, 25 bp, Artificial Sequence
  primer
cttcccgcagcactcgagcgatcgc

SEQ ID NO: 7 - DNA, 37 bp, Artificial Sequence
  primer
gtttaaacttaagcttgccaccatggcctttaatgac

SEQ ID NO: 8 - DNA, 25 bp, Artificial Sequence
  primer
aatggactctgactcgagcgatcgc

SEQ ID NO: 9 - DNA, 37 bp, Artificial Sequence
  primer
gtttaaacttaagcttgccaccatggctcagtttgtc

SEQ ID NO: 10 - DNA, 25 bp, Artificial Sequence
  primer
aagagctggaccctcgagcgatcgc

SEQ ID NO: 11 - DNA, 21 bp, Artificial Sequence
  primer
ctgtggcctttggatatctgt

SEQ ID NO: 12 - DNA, 20 bp, Artificial Sequence
  primer
acgtaggctaccagcagcag

SEQ ID NO: 13 - DNA, 18 bp, Artificial Sequence
  primer
cgacaggtttgggcggcg